(19) United States
(12) Patent Application Publication
(10) Pub. No.: US 2026/0114839 A1
(43) Pub. Date: Apr. 30, 2026

(54) ULTRASONIC DIAGNOSTIC APPARATUS

(71) Applicant: CANON MEDICAL SYSTEMS CORPORATION, Otawara-shi (JP)

(72) Inventors: Haruka SATO, Sakura (JP); Kuramitsu NISHIHARA, Otawara (JP); Akihiro KAKEE, Nasushiobara (JP)

(73) Assignee: CANON MEDICAL SYSTEMS CORPORATION, Otawara-shi (JP)

(21) Appl. No.: 19/368,109

(22) Filed: Oct. 24, 2025

(30) Foreign Application Priority Data
Oct. 29, 2024 (JP) 2024-190093

(51) Int. Cl. A61B 8/06; A61B 8/00

(52) U.S. Cl. CPC A61B 8/06; A61B 8/469; A61B 8/488; A61B 8/5207; A61B 8/5223; A61B 8/5292

(57) ABSTRACT

An ultrasonic diagnostic apparatus according to an embodiment includes a sample gate setting unit that sets a sample gate including a region where blood flow information of a subject is acquired on an ultrasonic image of the subject, an acquisition unit that acquires a plurality of blood vessel width candidates based on at least one of reception information of ultrasonic waves of the subject and examination information for a blood vessel at a position where the sample gate is set, and an adjustment unit that adjusts the region included in the sample gate based on the plurality of blood vessel width candidates.

### CROSS REFERENCE TO RELATED APPLICATIONS

This application is based upon and claims the benefit of priority from the prior Japanese Patent Application No. 2024-190093, filed on Oct. 29, 2024, the entire contents of which are incorporated herein by reference.

### FIELD

Embodiments described in the present specification and drawings relate to an ultrasonic diagnostic apparatus.

### BACKGROUND

Conventionally, in ultrasonic examination using an ultrasonic diagnostic apparatus, Doppler spectrum (Doppler waveform) representing blood flow information is displayed using Doppler information (Doppler signal) including a blood flow signal extracted from a reflected wave of an ultrasonic wave. The Doppler waveform is a waveform obtained by plotting the blood flow velocity at the position set by an operator as an observation site along the time series. For example, one of the methods for collecting the Doppler waveform is a pulsed wave Doppler (PWD) method. In a PWD mode (PWD scan) in which a Doppler waveform is collected using the PWD method, a user needs to set a sample gate including a region (sample volume) where blood flow information of a subject is acquired on an ultrasonic image (B mode image and color Doppler image), and manually set a sample volume according to a blood vessel width of a blood vessel at a position where the sample gate is set. Then, by the user setting an appropriate sample volume according to the blood vessel width of the blood vessel at the position where the sample gate is set, the accuracy of comparison of blood flow waveforms and measurement of the maximum blood flow and the blood flow rate is improved. For example, the size of a suitable sample volume depending on the blood vessel may be discussed in detail by examination guidelines.

However, since the width and shape of the blood vessel vary, when the sample volume is manually set for each blood vessel, the examination time may be long. In addition, in a case where blooming in which the blood flow signal is displayed to protrude from the blood vessel occurs in the color Doppler image displayed to be superimposed on the B mode image, since the B mode image of the portion where blooming occurs cannot be observed, it is difficult for the user to manually or subjectively adjust the sample volume, and in a case where such manual or subjective adjustment is performed, the accuracy of adjustment of the sample volume may decrease.

In recent years, there is a technique for recognizing a blood flow portion based on a color Doppler image superimposed and displayed on a B mode image and automatically adjusting the depth and width of a sample volume in accordance with the blood flow portion. However, in a case where blooming occurs in the color Doppler image superimposed and displayed on the B mode image, since the sample volume is not appropriately adjusted by recognizing a portion where blooming occurs as a blood flow portion, the accuracy of adjustment of the sample volume may be deteriorated similarly to manual or subjective adjustment. Further, when blooming occurs, it is difficult to observe the B mode image, and thus it is difficult for the user to manually adjust the sample volume after automatically adjusting the depth and width of the sample volume according to the blood flow portion. In addition, in a case where the adjustment of color gain and scale is not appropriately performed, there is a case where the color Doppler image does not exist up to the vicinity of a blood vessel wall, and it is difficult to accurately adjust the sample volume even in such a case. Therefore, it is desired to improve examination efficiency and examination accuracy in the PWD mode by appropriately adjusting the region where the blood flow information of the subject is acquired.

### BRIEF DESCRIPTION OF THE DRAWINGS

FIG. 1 is a block diagram illustrating an example of a configuration of an ultrasonic diagnostic apparatus according to a first embodiment;

FIG. 2 is a diagram illustrating an example of a sample gate set on an ultrasonic image;

FIG. 3 is a flowchart for describing blood vessel width candidate acquiring processing based on B mode information executed in an apparatus main body;

FIG. 4 is a diagram illustrating an example of an ultrasonic image in a certain range from a center position of a sample volume;

FIG. 5 is a diagram illustrating an example of distribution of brightness values in the certain range from the center position of the sample volume;

FIG. 6 is a diagram illustrating a distribution of brightness values in a case where a blood vessel is included in the certain range from the center position of the sample volume and a distribution of brightness values in a case where a uniform tissue is included in the certain range from the center position of the sample volume;

FIG. 7 is a diagram illustrating an example of distribution of brightness values on a raster;

FIG. 8 is a diagram for describing brightness values of a blood vessel, a tissue, and a blood vessel wall;

FIG. 9 is a diagram illustrating an example of a threshold acquired based on a distribution of brightness values of a B mode image in a certain range from a center position of a sample volume;

FIG. 10 is a diagram illustrating an example of distribution of brightness values on a raster in which a threshold is set;

FIG. 11 is a diagram for describing an example of a search method;

FIG. 12 is a diagram illustrating an example of a method of adjusting a sample volume;

FIG. 13 is a flowchart for describing sample volume width adjustment processing executed in the ultrasonic diagnostic apparatus according to the first embodiment;

FIG. 14 is a diagram illustrating an example of an ultrasonic image in which a sample gate is set;

FIG. 15 is a diagram illustrating an example of distribution of brightness values in a certain range from the center position of the sample volume acquired in the ultrasonic image illustrated in FIG. 14;

FIG. 16 is a diagram illustrating an example of distribution of brightness values on a raster acquired in the ultrasonic image illustrated in FIG. 14;

FIG. 17A is a diagram illustrating an example of an ultrasonic image in which a sample gate including a sample volume before adjustment is set;

FIG. 17B is a diagram illustrating an example of the ultrasonic image in which the sample gate including the adjusted sample volume is set;

FIG. 18 is a block diagram illustrating an example of a configuration of an ultrasonic diagnostic apparatus according to a second embodiment;

FIG. 19 is a flowchart for describing the blood vessel width candidate acquiring processing based on power information executed in the apparatus main body;

FIG. 20 is a diagram illustrating an example of a method of acquiring a power value;

FIG. 21 is a flowchart for describing sample volume width adjustment processing executed in the ultrasonic diagnostic apparatus according to the second embodiment;

FIG. 22 is a diagram illustrating an example of distribution of brightness values in a certain range from a center position of a sample volume acquired in an ultrasonic image;

FIG. 23 is a diagram illustrating an example of distribution of brightness values on a raster acquired in the ultrasonic image illustrated in FIG. 22;

FIG. 24 is a diagram illustrating an example of a distribution of power values on a raster;

FIG. 25A is a diagram illustrating an example of an ultrasonic image in which a sample gate including a sample volume before adjustment is set;

FIG. 25B is a diagram illustrating an example of an ultrasonic image in which a sample gate including sample volumes before and after adjustment is set;

FIG. 26 is a block diagram illustrating an example of a configuration of an ultrasonic diagnostic apparatus according to a third embodiment;

FIG. 27 is a flowchart for describing the blood vessel width candidate acquiring processing based on velocity information executed in the apparatus main body;

FIG. 28 is a flowchart for describing the blood vessel width candidate acquiring processing based on dispersion information executed in the apparatus main body;

FIG. 29 is a flowchart for describing sample volume width adjustment processing executed in the ultrasonic diagnostic apparatus according to the third embodiment;

FIG. 30 is a diagram illustrating an example of an ultrasonic image in which a sample gate is set;

FIG. 31 is a diagram illustrating an example of distribution of brightness values on a raster acquired in the ultrasonic image illustrated in FIG. 30;

FIG. 32A is a diagram illustrating an example of an ultrasonic image in which a sample gate including a sample volume before adjustment is set;

FIG. 32B is a diagram illustrating an example of an ultrasonic image in which a sample gate including sample volumes before and after adjustment is set;

FIG. 33 is a flowchart for describing sample volume width adjustment processing executed in the ultrasonic diagnostic apparatus according to the third embodiment;

FIG. 34 is a diagram illustrating an example of an ultrasonic image IM in which a sample gate is set;

FIG. 35 is a diagram illustrating an example of a distribution of power values in the ultrasonic image illustrated in FIG. 34;

FIG. 36 is a diagram illustrating an example of a distribution of velocity values in the ultrasonic image illustrated in FIG. 34;

FIG. 37 is a diagram illustrating an example of the distribution of power values on a raster illustrated in FIG. 35 and the distribution of velocity values on a raster illustrated in FIG. 36; and

FIG. 38 is a diagram obtained by normalizing each of the distribution of power values and the distribution of velocity values illustrated in FIG. 37.

### DETAILED DESCRIPTION

Hereinafter, respective embodiments of the ultrasonic diagnostic apparatus will be described with reference to the accompanying drawings. In the embodiments below, the same reference signs are given for identical components in terms of configuration and function, and duplicate description is omitted.

### First Embodiment

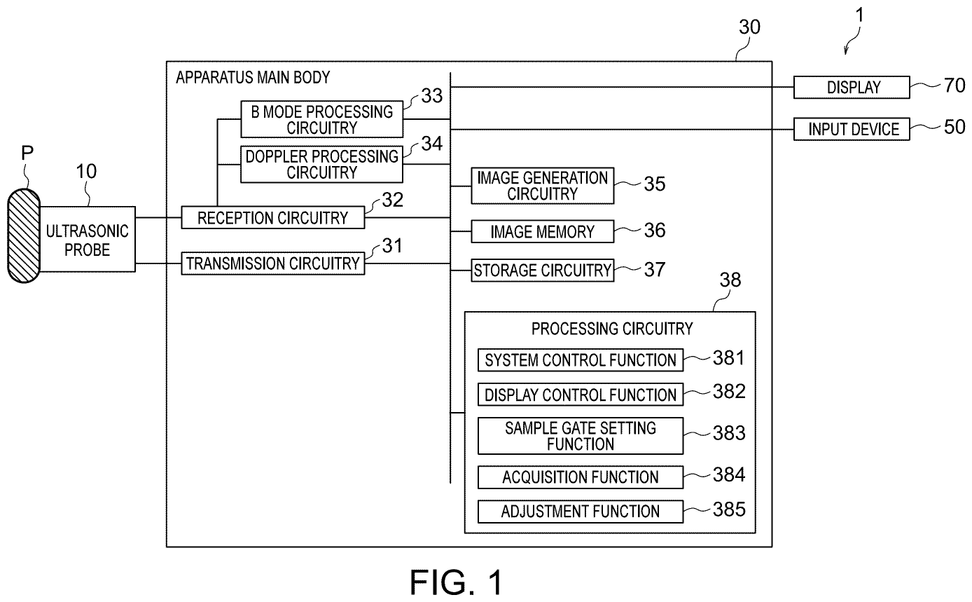

FIG. 1 is a block diagram illustrating an example of a configuration of an ultrasonic diagnostic apparatus according to a first embodiment. As illustrated in FIG. 1, an ultrasonic diagnostic apparatus 1 includes an ultrasonic probe 10, an apparatus main body 30, an input device 50, and a display 70.

The ultrasonic probe 10 includes, for example, a plurality of piezoelectric vibrators. The plurality of piezoelectric vibrators generate ultrasonic waves based on a drive signal supplied from a transmission circuitry 31 included in the apparatus main body 30 to be described later. In addition, the ultrasonic probe 10 receives a reflected wave from a subject P and converts the reflected wave into an electrical signal. In addition, the ultrasonic probe 10 includes, for example, a matching layer provided on the piezoelectric vibrator, a backing material for preventing propagation of an ultrasonic wave rearwards from the piezoelectric vibrator and the like.

When ultrasonic waves are transmitted from the ultrasonic probe 10 to the subject P, the transmitted ultrasonic waves are reflected one after another on a discontinuous surface of an acoustic impedance in a body tissue of the subject P, and are received as a reflected wave signal by the plurality of piezoelectric vibrators included in the ultrasonic probe 10. The amplitude of the received reflected wave signal depends on the difference in acoustic impedance at the discontinuous surface at which ultrasonic waves are reflected. Note that a reflected wave signal in a case where transmitted ultrasonic pulses are reflected by a moving blood flow or a surface of a heart wall or the like receives a frequency shift depending on a velocity component with respect to the direction of ultrasonic transmission of a moving body, due to the Doppler effect. This reflected wave signal corresponds to a reception signal in the present embodiment.

The ultrasonic probe 10 is detachably connected to the apparatus main body 30. In a case where scanning a two-dimensional region in the subject P (two-dimensional scanning) is performed, the user connects, for example, a 1D array probe in which a plurality of piezoelectric vibrators are arranged in a line to the apparatus main body 30 as the ultrasonic probe 10. The 1D array probe is a linear type ultrasonic probe, a convex type ultrasonic probe, a sector type ultrasonic probe, or the like. Further, in a case where scanning a three-dimensional region in the subject P (three-dimensional scanning) is performed, the user connects, for example, a mechanical 4D probe or a 2D array probe as the ultrasonic probe 10 to the apparatus main body 30. The mechanical 4D probe can perform two-dimensional scanning using a plurality of piezoelectric vibrators arranged in a line like the 1D array probe, and can perform three-dimensional scanning by swinging the plurality of piezoelectric vibrators at a predetermined angle (a swing angle). In addition, a 2D array probe can perform three-dimensional scanning by a plurality of piezoelectric vibrators arranged in a matrix, and can perform two-dimensional scanning by focusing and transmitting ultrasonic waves. Note that the 2D array probe can simultaneously perform two-dimensional scanning of a plurality of cross sections.

Further, the ultrasonic diagnostic apparatus 1 according to the present embodiment collects Doppler waveforms by a PWD method. Therefore, in the present embodiment, the ultrasonic probe 10 connected to the apparatus main body 30 is an ultrasonic probe capable of executing ultrasonic wave transmission/reception for capturing a two-dimensional ultrasonic image and ultrasonic wave transmission/reception for collecting a Doppler waveform by a PWD method.

The apparatus main body 30 generates an ultrasonic image based on a reflected wave signal received by the ultrasonic probe 10. Specifically, the apparatus main body 30 can generate a two-dimensional ultrasonic image based on a reflected wave signal corresponding to a two-dimensional region of the subject P received by the ultrasonic probe 10. In addition, the apparatus main body 30 can generate a three-dimensional ultrasonic image based on a reflected wave signal corresponding to a three-dimensional region of the subject P received by the ultrasonic probe 10. As illustrated in FIG. 1, the apparatus main body 30 includes the transmission circuitry 31, a reception circuitry 32, a B mode processing circuitry 33, a Doppler processing circuitry 34, an image generation circuitry 35, an image memory 36, a storage circuitry 37, and a processing circuitry 38.

The transmission circuitry 31 includes a pulse generator, a transmission delay unit, a pulser, and the like, and supplies a drive signal to the ultrasonic probe 10. The pulse generator repeatedly generates rate pulses for forming transmission ultrasonic waves at a predetermined rate frequency. Further, the transmission delay unit focuses ultrasonic waves generated from the ultrasonic probe 10 into a beam shape, and gives, to each rate pulse generated by the pulse generator, a delay time for each piezoelectric vibrator necessary to determine transmission directivity. Further, the pulser applies a drive signal (drive pulse) to the ultrasonic probe 10 at a timing based on the rate pulse. That is, the transmission delay unit arbitrarily adjusts the transmission direction of the ultrasonic wave transmitted from the piezoelectric vibrator surface by changing the delay time given to each rate pulse.

Note that the transmission circuitry 31 has a function of instantaneously changing a transmission frequency, a transmission driving voltage, and the like in order to execute a predetermined scan sequence based on an instruction from the processing circuitry 38 to be described later. In particular, the transmission driving voltage is changed by a linear amplifier type transmission circuitry capable of instantaneously switching the value thereof or a mechanism for electrically switching a plurality of power supply units.

The reception circuitry 32 includes a preamplifier, an analog/digital (A/D) converter, a reception delay unit, an adder, and the like, and performs various types of processing on the reflected wave signal received by the ultrasonic probe 10, thereby generating reflected wave data. The preamplifier amplifies the reflected wave signal for each channel and performs gain adjustment (gain correction). The A/D converter A/D converts the gain-corrected reflected wave signal into a digital signal by A/D converting the gain-corrected reflected wave signal. The reception delay unit gives a delay time necessary to determine reception directivity. The adder performs processing of adding reflected wave signals processed by the reception delay unit, and generates reflected wave data. Then, the adder outputs the generated reflected wave data to the B mode processing circuitry 33 and the Doppler processing circuitry 34.

In a case where the subject P is two-dimensionally scanned, the transmission circuitry 31 causes the ultrasonic probe 10 to transmit a two-dimensional ultrasonic beam. Then, the reception circuitry 32 generates two-dimensional reflected wave data from a two-dimensional reflected wave signal received by the ultrasonic probe 10. Further, in a case where the subject P is three-dimensionally scanned, the transmission circuitry 31 according to the present embodiment causes the ultrasonic probe 10 to transmit a three-dimensional ultrasonic beam. Then, the reception circuitry 32 generates three-dimensional reflected wave data from a three-dimensional reflected wave signal received by the ultrasonic probe 10.

The B mode processing circuitry 33 receives the reflected wave data from reception circuitry 32, performs logarithmic amplification, envelope detection processing, and the like, and generates data (B mode data) in which signal intensity is expressed by brightness level.

The Doppler processing circuitry 34 performs frequency analysis on the reflected wave data received from the reception circuitry 32, extracts blood flow, tissue, and contrast medium echo components due to the Doppler effect in a region of interest (ROI) set in the scan region, and generates data (Doppler data) obtained by extracting moving body information such as velocity, dispersion, and power for multiple points. Specifically, the Doppler processing circuitry 34 generates Doppler data in which a velocity value, a dispersion value, a power value, and the like are estimated at each of the plurality of sample points as the motion information of the moving body. Here, the moving body is, for example, a blood flow, a tissue of an organ that periodically moves such as a heart wall, or a contrast medium. The Doppler processing circuitry 34 outputs the generated Doppler data to the image generation circuitry 35 and the image memory 36.

In addition, the Doppler processing circuitry 34 can execute a color Doppler method also called a color flow mapping (CFM) method. In the color flow mapping method, ultrasonic waves are transmitted and received a plurality of times on a plurality of rasters. In the color flow mapping method, a moving target indicator (MTI) filter is applied to a data string at the same position to suppress a signal (clutter signal) derived from a stationary tissue or a slow-moving tissue from the data string at the same position and extract a blood flow signal which is a signal derived from blood flow. In the color flow mapping method, blood flow information such as a velocity value of blood flow, a dispersion value of blood flow, and a power value of blood flow is estimated from the blood flow signal. The Doppler processing circuitry 34 outputs color Doppler data indicating blood flow information estimated by the color flow mapping method to the image generation circuitry 35 and the image memory 36. Note that the color Doppler data is an example of Doppler data.

The MTI filter outputs a data string in which a clutter component is suppressed and a blood flow signal is extracted from the data string of continuous reflected wave data at the same position (same sample point) using a filter matrix. As the MTI filter, for example, a filter in which a coefficient is fixed, such as a Butterworth infinite impulse response (IIR) filter or a polynomial regression filter, or an adaptive filter in which a coefficient is changed according to an input signal using an eigenvector or the like can be applied.

The B mode processing circuitry 33 and the Doppler processing circuitry 34 can process both two-dimensional reflected wave data and three-dimensional reflected wave data. That is, the B mode processing circuitry 33 generates two-dimensional B mode data from two-dimensional reflected wave data, and generates three-dimensional B mode data from three-dimensional reflected wave data. In addition, the Doppler processing circuitry 34 generates two-dimensional Doppler data from two-dimensional reflected wave data and generates three-dimensional Doppler data from three-dimensional reflected wave data.

The image generation circuitry 35 generates an ultrasonic image expressed in a predetermined brightness range based on a reflected wave signal received by the ultrasonic probe 10. For example, the image generation circuitry 35 generates, as the ultrasonic image, a two-dimensional B mode image in which the intensity of the reflected wave is represented by brightness from the two-dimensional B mode data generated by the B mode processing circuitry 33. In addition, the image generation circuitry 35 generates, as an ultrasonic image, a velocity image, a distributed image, a power image, or a two-dimensional color Doppler image as a combination image thereof, representing the moving body information from the two-dimensional Doppler data generated by the Doppler processing circuitry 34.

Here, the image generation circuitry 35 generally converts (scan converts) a scanning line signal sequence of ultrasonic scanning into a scanning line signal sequence of a video format represented by a television or the like, and generates a display ultrasonic image. For example, the image generation circuitry 35 generates the display ultrasonic image by performing coordinate conversion according to a scanning mode of an ultrasonic wave by the ultrasonic probe 10. In addition, for example, the image generation circuitry 35 performs, as various types of image processing other than the scan conversion, image processing (smoothing processing) of regenerating an average value image of brightness by using a plurality of image frames after scan conversion, image processing (edge enhancement processing) using a differential filter in the image, and the like.

That is, the B mode data and the Doppler data are data before scan conversion processing, and the data generated by the image generation circuitry 35 is a display ultrasonic image after the scan conversion processing. Note that the B mode data and the Doppler data, which are data before the scan conversion processing, are also referred to as raw data. The image generation circuitry 35 generates the two-dimensional B mode image or the two-dimensional color Doppler image, which is a two-dimensional ultrasonic image, from the two-dimensional B mode data or the two-dimensional Doppler data, which is raw data before the scan conversion processing. Furthermore, the image generation circuitry 35 can also generate, for example, a superimposed image in which a color Doppler image is superimposed on the two-dimensional B mode image. Note that the image generation circuitry 35 can also generate a three-dimensional B mode image or a three-dimensional color Doppler image by performing coordinate transformation on three-dimensional B mode data generated by the B mode processing circuitry 33 and three-dimensional Doppler data generated by the Doppler processing circuitry 34, which are raw data before the scan conversion processing.

Furthermore, for example, the image generation circuitry 35 generates an M-mode image from the time-series data of the B mode data on one scanning line generated by the B mode processing circuitry 33. In addition, the image generation circuitry 35 generates a Doppler waveform obtained by plotting a blood flow and tissue velocity information along a time series from the Doppler data generated by the Doppler processing circuitry 34.

The image memory 36 is a memory that stores various images generated by the image generation circuitry 35. The image memory 36 also stores data generated by the B mode processing circuitry 33 and the Doppler processing circuitry 34. The B mode data and the Doppler data stored in the image memory 36 can be called by an operator after diagnosis, for example, and become a display ultrasonic image when passing through the image generation circuitry 35. Further, the image memory 36 also stores the reflected wave data output from the reception circuitry 32. For example, the image memory 36 is realized by a random access memory (RAM), a semiconductor memory element such as a flash memory, a hard disk, or an optical disk.

The storage circuitry 37 stores various types of data such as a control program for performing ultrasonic wave transmission/reception, image processing, and display processing, diagnostic information (for example, patient ID, doctor's finding, and the like), a diagnostic protocol, and various body marks. In addition, the storage circuitry 37 stores a first relation table in which the examination information and the blood vessel diameter are associated with each other. In addition, the storage circuitry 37 stores information regarding the setting of the sample gate. The information regarding the setting of the sample gate stores, for example, information such as the width of the sample volume included in the sample gate before adjustment and the angle of the scanning line (raster). In addition, the storage circuitry 37 is also used to store data stored in the image memory 36, as necessary. For example, the storage circuitry 37 is realized by a semiconductor memory element such as a flash memory, a hard disk, or an optical disk.

The processing circuitry 38 executes various types of data processing. The processing circuitry 38 has a system control function 381, a display control function 382, a sample gate setting function 383, an acquisition function 384, and an adjustment function 385. Here, for example, each processing function of the system control function 381, the display control function 382, the sample gate setting function 383, the acquisition function 384, and the adjustment function 385, which are components of the processing circuitry 38 illustrated in FIG. 1, is recorded in the storage circuitry 37 in the form of a program executable by a computer. The processing circuitry 38 reads each program from the storage circuitry 37 and executes each read program to implement a function corresponding to each program. In other words, the processing circuitry 38 in a state of reading each program has each function illustrated in the processing circuitry 38 of FIG. 1. The processing circuitry 38 is realized by, for example, a processor. The system control function 381 is an example of a control unit. The display control function 382 is an example of a display control unit. The sample gate setting function 383 is an example of a sample gate setting unit. The acquisition function 384 is an example of an acquisition unit. The adjustment function 385 is an example of an adjustment unit.

The system control function 381 is a function of integrally controlling the entire operation of the ultrasonic diagnostic apparatus 1. For example, the system control function 381 controls the transmission circuitry 31 and the reception circuitry 32 based on parameters related to transmission and reception of ultrasonic waves according to various modes. The various modes include, for example, a B mode, a color Doppler mode, a PWD mode, a CWD mode, and the like. Note that the PWD mode is also referred to as a PW mode, and the CWD mode is also referred to as a CW mode.

The B mode is a mode for generating a B mode image by B mode scanning. The color Doppler mode is a mode for generating a color Doppler image in which a color is allocated to blood flow information measured using, for example, a pulse wave by color Doppler mode scanning. The color Doppler mode scanning includes B mode scanning. Then, in the color Doppler mode, for example, both the B mode image and the color Doppler image are generated, and the color Doppler image is superimposed and displayed on the B mode image.

The PWD mode is a mode for collecting a Doppler waveform related to a specific measurement site by a PWD mode scan (PWD method scan) for transmitting a pulse wave to a subject and receiving a reflected wave. In the PWD mode, it is common for the ultrasonic probe 10 to perform the PWD mode scan on one raster, but it is also possible to perform the PWD mode scan on a plurality of rasters. In this case, the ultrasonic probe 10 sequentially transmits pulse waves to the plurality of rasters and receives reflected waves. Note that, at the time of execution of the PWD mode, only the Doppler waveform may be updated in order to observe the blood flow with high image quality, that is, the B mode scan need not be used in combination, and the B mode image and the Doppler waveform may be updated in order to compare with the B mode image, that is, the B mode scan may be used in combination.

The CWD mode is a mode for measuring a Doppler waveform on one raster by CWD mode scanning (scanning of a CWD method) for receiving a reflected wave while transmitting a continuous wave. In the CWD mode, it is necessary to continuously apply the continuous wave to a target, and thus, the B mode scan cannot be used together.

The display control function 382 is a function of controlling the display 70 so as to display various ultrasonic images, Doppler waveforms, and the like generated by the image generation circuitry 35. For example, the display control function 382 controls the display 70 to display the B mode image generated by the image generation circuitry 35, the color Doppler image, or an image including both of them, and the Doppler waveform. Specifically, the display control function 382 displays the B mode image, the color Doppler image, or the image including both of them live on the display 70 (in real time), or displays the Doppler waveform live on the display 70.

The sample gate setting function 383 sets a sample gate including a region for acquiring blood flow information of a subject on an ultrasonic image of the subject. Specifically, the sample gate setting function 383 sets a sample gate including a region where blood flow information of the subject is acquired on the ultrasonic image of the subject in the B mode image as the ultrasonic image or the color Doppler image superimposed and displayed on the B mode image according to an input operation of the user. In addition, the sample gate setting function 383 sets a sample gate in an ultrasonic image obtained by imaging a blood vessel from one imaging direction. The region where the blood flow information is acquired is, for example, a sample volume. In the following description, the embodiment will be described assuming that the region where the blood flow information is acquired is the sample volume.

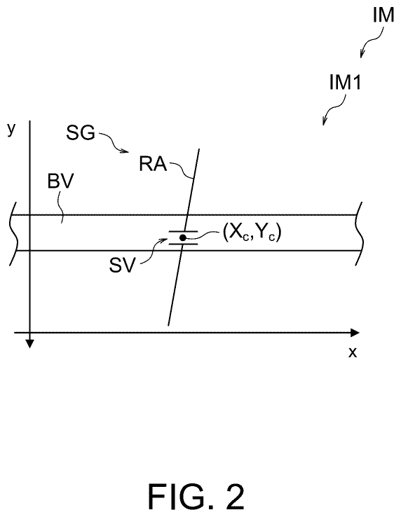

In addition, the sample gate setting function 383 acquires the center position of the region included in the sample gate. Specifically, the sample gate setting function 383 acquires the center position of the sample volume included in the sample gate. FIG. 2 is a diagram illustrating an example of a sample gate set on an ultrasonic image. An ultrasonic image IM illustrated in FIG. 2 is a B mode image IM1. As illustrated in FIG. 2, the sample gate setting function 383 sets the sample gate SG including the sample volume SV on the ultrasonic image IM. Specifically, in the example illustrated in FIG. 2, the sample gate setting function 383 sets the sample gate SG in the B mode image IM1, which is the ultrasonic image IM in which a long axis image of a blood vessel BV is captured, as the ultrasonic image IM obtained by imaging the blood vessel BV from one imaging direction. In the example illustrated in FIG. 2, the sample volume SV included in the sample gate SG is set on the blood vessel BV depicted in the ultrasonic image IM. The sample gate setting function 383 acquires coordinates (X_{c}, Y_{c}) of the center position of the sample volume SV included in the sample gate SG.

In addition, the sample gate setting function 383 receives a first input operation related to adjustment of the sample volume from the user. The first input operation related to the adjustment of the sample volume is an input operation for starting the adjustment of the sample volume, and is, for example, an operation of pressing a sample volume width adjustment button included in the input device 50.

The acquisition function 384 acquires, based on at least one of the reception information of ultrasonic waves of the subject and the examination information, a plurality of blood vessel width candidates for the blood vessel at the position where the sample gate SG is set. Specifically, the acquisition function 384 acquires, based on at least one of the reception information of ultrasonic waves of the subject and the examination information, a plurality of blood vessel width candidates for the blood vessel BV in the ultrasonic image IM obtained by imaging the blood vessel BV from one imaging direction. For example, in the example illustrated in FIG. 2, the acquisition function 384 acquires, based on at least one of the reception information of ultrasonic waves of the subject and the examination information a plurality of blood vessel width candidates, as the blood vessel BV in the ultrasonic image IM obtained by imaging the blood vessel BV from one imaging direction, for the blood vessel BV in the B mode image IM1 which is the ultrasonic image IM in which the long axis image of the blood vessel BV is captured.

The reception information of ultrasonic waves is information regarding the reflected wave signal received by the ultrasonic probe 10. The reception information of ultrasonic waves is information such as B mode data and Doppler data which are raw data before the scan conversion processing, and an ultrasonic image for display after the scan conversion processing. Specifically, the reception information of ultrasonic waves includes, for example, power information regarding power of blood flow based on a reflected wave signal of the ultrasonic wave, dispersion information regarding dispersion of blood flow based on the reflected wave signal, velocity information regarding velocity of blood flow based on the reflected wave signal, and B mode information regarding B mode based on the reflected wave signal. The power information includes, for example, information related to a power value in Doppler data, power of blood flow in color Doppler data, a power image, and the like. The dispersion information includes, for example, information regarding a dispersion value in Doppler data, dispersion of blood flow in color Doppler data, a distributed image, and the like. The velocity information includes, for example, information regarding a velocity value in Doppler data, a velocity value of blood flow in color Doppler data, a velocity image, and the like. The B mode information includes, for example, information related to distribution of brightness values of the B mode data and the B mode image. The examination information is information related to ultrasonic examination. The examination information includes, for example, setting contents of the examination mode, Depth (view depth), and a depth and a size of a region of interest (ROI). The setting content of the examination mode includes information of examination sites such as the carotid artery, the abdomen, and the lower limb blood vessels.

Therefore, the acquisition function 384 acquires, based on at least any two pieces of information among the power information, the dispersion information, the velocity information, the B mode information, and the examination information, blood vessel width candidates based on each of at the least any two pieces of information as a plurality of blood vessel width candidates. In the following description, it is assumed that the acquisition function 384 according to the present embodiment acquires, based on each of the examination information and the B mode information which are the examination information and one piece of the reception information of ultrasonic waves, two blood vessel width candidates based on each of the examination information and the B mode information.

First, an example of a method of acquiring a blood vessel width candidate based on examination information will be described. The acquisition function 384 acquires the examination information from the storage circuitry 37. Then, when the examination information includes the carotid artery as the setting content of the examination mode, the acquisition function 384 acquires the blood vessel diameter of the carotid artery as the blood vessel width candidate from the first relation table stored in the storage circuitry 37 based on the examination information. Note that, in the above example, the case where the examination information includes the carotid artery has been described, but the examination site is not limited to the carotid artery. That is, the examination site is arbitrary, and for example, the examination information may include an abdomen or the like. Even when the examination information includes an abdomen or the like, the acquisition function 384 acquires a blood vessel diameter corresponding to the examination site from the first relation table as a blood vessel width candidate. Note that, when the blood vessel width candidate cannot be acquired based on the examination information, an error value may be stored in the blood vessel width candidate based on the examination information.

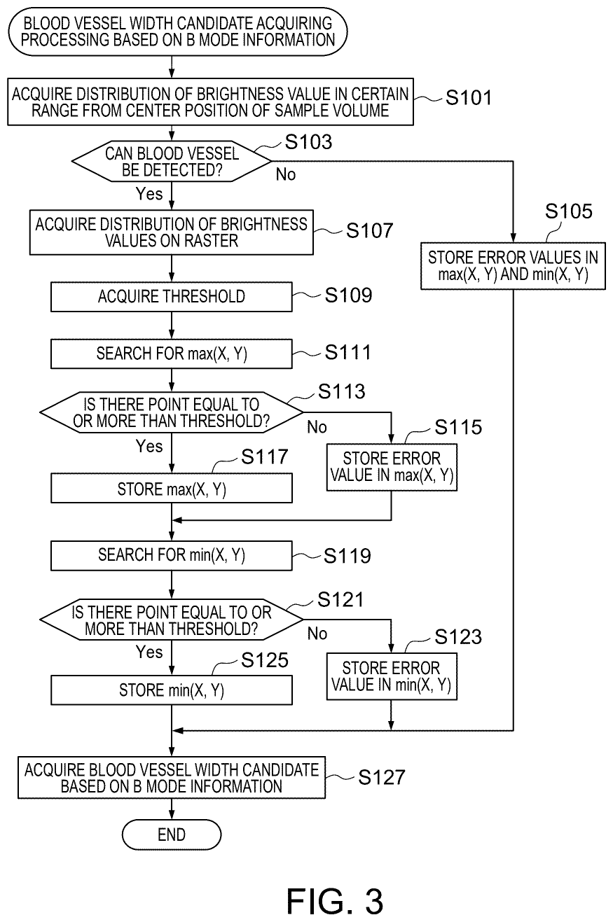

Next, a method for acquiring a blood vessel width candidate based on B mode information will be described with reference to FIG. 3. FIG. 3 is a flowchart for describing blood vessel width candidate acquiring processing based on B mode information executed in the apparatus main body 30. In the blood vessel width candidate acquiring processing based on the B mode information illustrated in FIG. 3, a distribution of brightness values in a certain range is acquired using the B mode image IM1, whether or not a blood vessel is detected is determined, a distribution of brightness values on a raster is acquired, a threshold is acquired, max(X, Y) and min(X, Y) are searched for, and a blood vessel width candidate based on the B mode information is acquired.

As illustrated in FIG. 3, first, the acquisition function 384 in the processing circuitry 38 of the apparatus main body 30 acquires a distribution of brightness values of the B mode image IM1 in a certain range from the center position of the sample volume SV (step S101). Specifically, the acquisition function 384 acquires a distribution of brightness values in the certain range from the center position of the sample volume SV acquired by the sample gate setting function 383 in the B mode image IM1 which is the ultrasonic image IM.

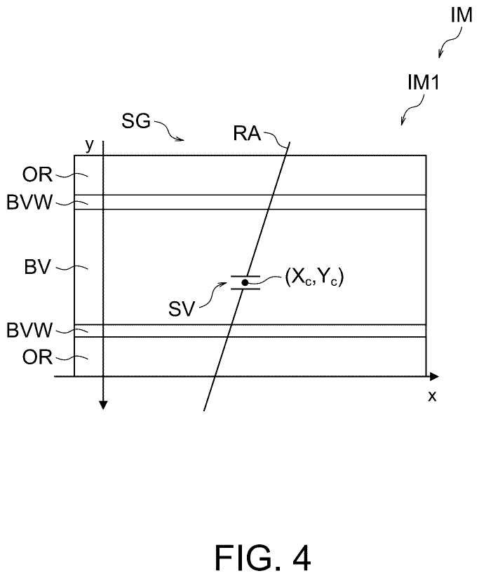

FIG. 4 is a diagram illustrating an example of an ultrasonic image IM in the certain range from the center position of the sample volume SV. In the example illustrated in FIG. 4, the ultrasonic image IM is a B mode image IM1. In addition, in the example illustrated in FIG. 4, a blood vessel BV, a blood vessel wall BVW, and a tissue OR are drawn in a B mode image IM1 which is an ultrasonic image IM.

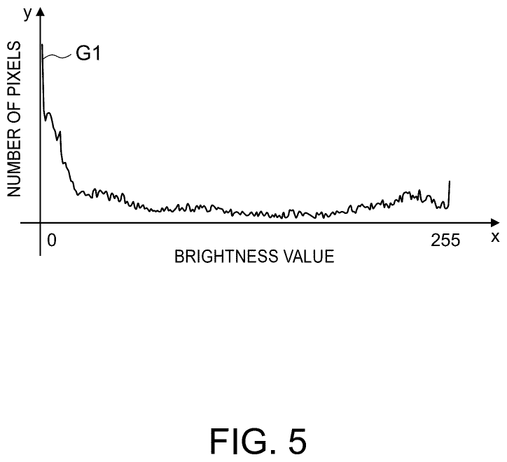

FIG. 5 is a diagram illustrating an example of distribution of brightness values in the certain range from the center position of the sample volume SV. As illustrated in FIG. 5, the acquisition function 384 acquires a brightness value for each pixel in the B mode image IM1, which is the ultrasonic image IM, in the certain range from the center position of the sample volume SV as a distribution of brightness values in the certain range from the center position of the sample volume SV, and acquires a graph G1 in which the X-axis is a brightness value and the Y-axis is the number of pixels.

Next, as illustrated in FIG. 3, the acquisition function 384 determines whether or not the blood vessel BV can be detected (step S103). Specifically, the acquisition function 384 determines whether or not the blood vessel BV can be detected from the distribution of the brightness values acquired in step S101.

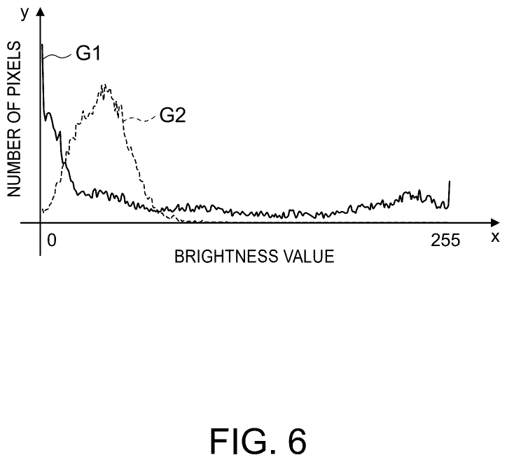

A method for determining the blood vessel detection will be described with reference to FIG. 6. FIG. 6 is a diagram illustrating a distribution of brightness values in a case where the blood vessel BV is included in the certain range from the center position of the sample volume SV, and a distribution of brightness values in a case where a uniform tissue is included in the certain range from the center position of the sample volume SV. A graph G1 illustrated in FIG. 6 is a distribution of brightness values in a case where a blood vessel BV such as a carotid artery is included, and a graph G2 illustrated in FIG. 6 is a distribution of brightness values in a case where a uniform tissue such as an abdomen is included. When an average value and a median value of each of the graph G1 and the graph G2 are calculated, the difference between the median value and the average value increases in the graph G1, whereas the difference between the median value and the average value decreases in the graph G2. That is, when the blood vessel BV is included in the certain range from the center position of the sample volume SV, the difference between the average value and the median value in the distribution of the brightness values increases. Therefore, as a method of determining blood vessel detection, the acquisition function 384 calculates an average value and a median value of the distribution of the brightness values acquired in step S101, and determines whether or not the blood vessel BV can be detected by determining whether or not a difference between the average value and the median value is equal to or more than a predetermined threshold.

Note that the acquisition function 384 may determine whether or not the blood vessel BV can be detected by determining whether or not the difference between the average value and the median value is equal to or more than a predetermined threshold, and further comparing the average value of the distribution of the brightness values with the brightness value at the center position of the sample volume even when it is determined that the difference between the average value and the median value is equal to or more than the predetermined threshold. In this case, when it is determined that the difference between the average value and the median value is equal to or more than the predetermined threshold, the acquisition function 384 compares the average value of the distribution of the brightness values with the brightness value at the center position of the sample volume SV, determines that the blood vessel BV can be detected when the brightness value at the center position of the sample volume SV is lower than the average value of the distribution of the brightness values, and determines that the blood vessel BV cannot be detected when the brightness value at the center position of the sample volume SV is higher than the average value of the distribution of the brightness values.

Then, in step S103, when the blood vessel BV cannot be detected (step S103: No), the acquisition function 384 stores error values in max(X, Y) and min(X, Y) (step S105).

On the other hand, in step S103, when the blood vessel BV can be detected (step S103: Yes), the acquisition function 384 acquires the distribution of the brightness value on a raster RA (step S107). Specifically, the acquisition function 384 acquires the distribution of the brightness value on the raster RA in the sample gate SG based on the ultrasonic image IM in which the sample gate SG is set.

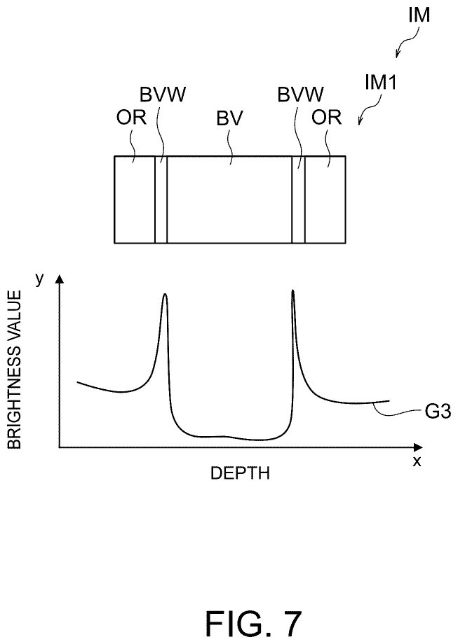

FIG. 7 is a diagram illustrating an example of distribution of brightness values on the raster RA. In the example illustrated in FIG. 7, the acquisition function 384 acquires a graph G3 in which the x-axis is Depth and the y-axis is a brightness value as the distribution of the brightness values on the raster RA. As illustrated in FIG. 5, the brightness value of the graph G3 decreases at Depth corresponding to the blood vessel BV and increases at Depth corresponding to the blood vessel wall BVW.

Next, as illustrated in FIG. 3, the acquisition function 384 acquires a threshold (step S109). Specifically, the acquisition function 384 acquires the threshold regarding the brightness value based on the distribution of the brightness value of the B mode image IM1 in a certain range from the center position of the sample volume SV acquired in step S101.

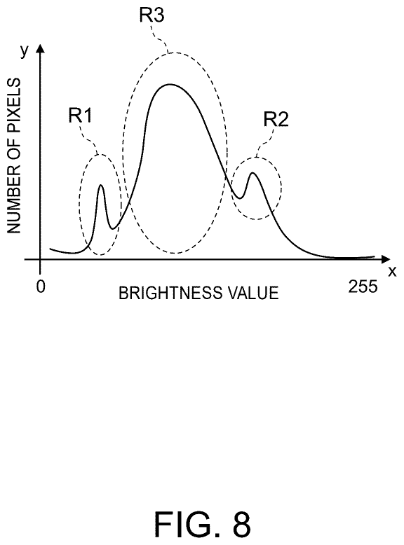

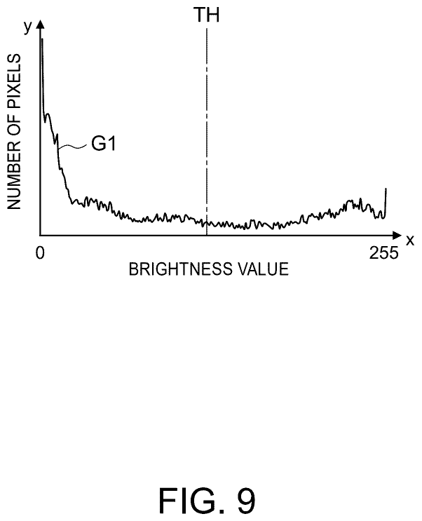

A method of acquiring the threshold will be described with reference to FIGS. 8 and 9. FIG. 8 is a diagram for describing brightness values of a blood vessel, a tissue, and a blood vessel wall. FIG. 9 is a diagram illustrating an example of a threshold acquired based on the distribution of the brightness values of the B mode image IM1 in a certain range from the center position of the sample volume SV. In the graph illustrated in FIG. 8 in which the x-axis is the brightness value and the y-axis is the number of pixels, a region R1 having a small brightness value can be estimated as a blood vessel, a region R2 having a high brightness value can be estimated as a blood vessel wall, and a region R3 between R1 and R2 can be estimated as a tissue. Therefore, as illustrated in FIG. 9, the acquisition function 384 estimates the brightness value indicating the blood vessel wall BVW in the distribution of the brightness values based on the graph G1 that is the distribution of the brightness values in a certain range from the center position of the sample volume SV, and acquires the brightness value indicating the estimated blood vessel wall BVW as a threshold TH.

Note that, since the distribution of the brightness values in a certain range from the center position of the sample volume SV varies according to setting information of the device such as the gain, the threshold TH acquired in step S109 also varies according to the setting information of the device such as the gain. That is, the acquisition function 384 may acquire the setting information and acquire the threshold TH based on the setting information and the distribution of the brightness value of the B mode image IM1 in a certain range from the center position of the sample volume SV.

Next, as illustrated in FIG. 3, the acquisition function 384 searches for max(X, Y) (step S111). Specifically, the acquisition function 384 sets the threshold acquired in step S109 to the distribution of brightness values on the raster RA acquired in step S107, moves a point along the raster RA in a direction in which Depth increases from the center position of the sample volume SV, and searches for a position where the brightness value of the point at each position is equal to or more than the threshold TH, thereby searching for max(X, Y).

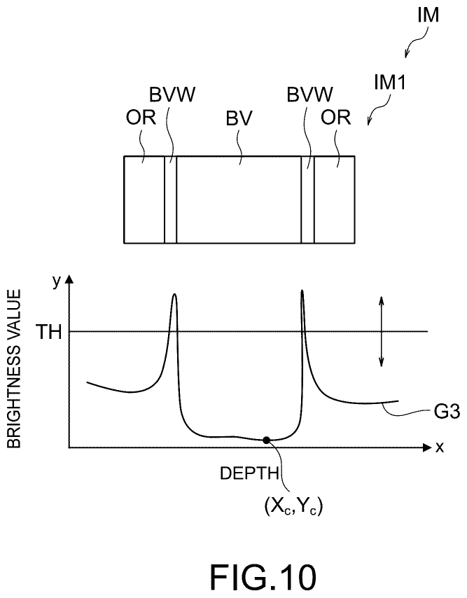

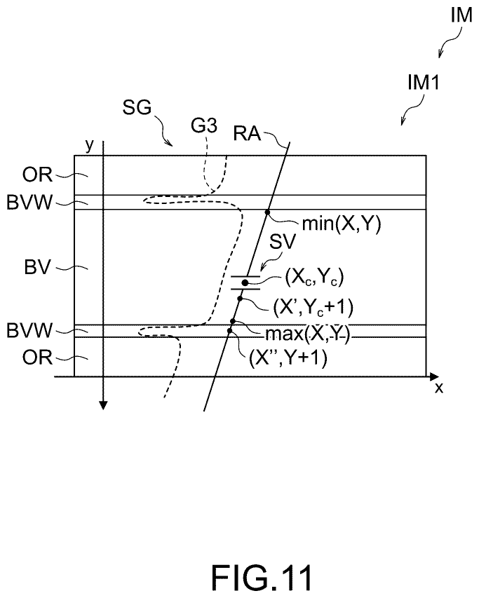

A method of searching for max(X, Y) will be described with reference to FIGS. 10 and 11. FIG. 10 is a diagram illustrating an example of a distribution of brightness values on the raster RA in which the threshold TH is set. FIG. 11 is a diagram for describing an example of a search method. As illustrated in FIG. 10, the acquisition function 384 sets the threshold acquired in step S109 to the distribution of the brightness value on the raster RA acquired in step S107. Then, as illustrated in FIG. 11, the acquisition function 384 moves the point along the raster RA in a direction in which the depth increases from the coordinates (X_{c}, Y_{c}) of the center position of the sample volume, that is, in the positive direction of the Y-axis illustrated in FIG. 11, and acquires the brightness value of the point of the coordinates (X′, Y_{c}+1). Then, the acquisition function 384 searches for a point at which the brightness value is equal to or more than the threshold TH by repeatedly moving the point along the raster RA in the positive direction of the Y-axis illustrated in FIG. 11 and acquiring the brightness value of each point. Note that, in the example illustrated in FIG. 11, the graph G3 illustrated in FIG. 10 is superimposed on the ultrasonic image IM, but the graph G3 need not be superimposed on the ultrasonic image IM.

Next, as illustrated in FIG. 3, the acquisition function 384 determines whether or not there is a point at which it is equal to or more than the threshold TH (step S113). Specifically, the acquisition function 384 determines whether or not there is a point at which the brightness value is equal to or more than the threshold TH based on the search result in step S111. More specifically, for example, the acquisition function 384 determines whether or not there is a point at which the brightness value is equal to or more than the threshold TH based on whether or not points at which the brightness value is equal to or more than the threshold TH continue. Then, in step S113, when there is no point at which it is equal to or more than the threshold TH (step S113: No), the acquisition function 384 in the processing circuitry 38 of the apparatus main body 30 stores an error value in max(X, Y) (step S115).

Note that the acquisition function 384 determines whether or not there is a point at which the brightness value is equal to or more than the threshold TH by determining whether or not a point at which the brightness value is equal to or more than the threshold TH continues once, but it is not limited thereto. That is, a method of determining whether or not there is a point at which the brightness value is equal to or more than the threshold TH is arbitrary. For example, the acquisition function 384 may determine whether or not there is a point at which the brightness value is equal to or more than the threshold TH by determining whether or not there are consecutive points at which the brightness value is equal to or more than the threshold TH twice or more, or may determine whether or not there is a point at which the brightness value is equal to or more than the threshold TH by determining whether or not a point at which the brightness value is equal to or more than the threshold TH has been searched once.

On the other hand, in step S113, when there is a point at which the brightness value is equal to or more than the threshold TH (step S113: Yes), the acquisition function 384 stores max(X, Y) (step S117). Specifically, the acquisition function 384 stores, as max(X, Y), coordinates of a point moved back a predetermined value in the negative direction of the y-axis from a point at which the brightness value is equal to or more than the threshold TH. More specifically, for example, the acquisition function 384 stores, as max(X, Y), the coordinates of a point where the y coordinate is moved back only 1 from the coordinates of the first point at which the brightness value becomes equal to or more than the threshold TH when the points at which the brightness value is equal to or more than the threshold TH are continuous along the raster RA. For example, in the example illustrated in FIG. 11, since the brightness value of the point of the coordinates (X″, Y+1) and the brightness value of the point of the coordinates (X′″, Y+2) continuously become equal to or more than the threshold TH, the acquisition function 384 stores, as max(X, Y), (X′, Y) where the y coordinate is moved back only 1 from the coordinates (X″, Y+1) of the first point at which the brightness value becomes equal to or more than the threshold TH along the raster RA.

Note that although the acquisition function 384 stores, as max(X, Y), the coordinates of a point where the y coordinate is moved back only 1 from the coordinates of a point at which the brightness value is equal to or more than the threshold TH, it is not limited thereto. That is, the coordinates stored as max(X, Y) by the acquisition function 384 are arbitrary, and for example, the acquisition function 384 may store, as max(X, Y), the coordinates of a point one or more points before the point at which the brightness value is equal to or more than the threshold TH, or may store, as max(X, Y), the coordinates of a point moved by a predetermined value from the point at which the brightness value is equal to or more than the threshold TH, based on the search result in step S11.

Next, as illustrated in FIG. 3, the acquisition function 384 in the processing circuitry 38 of the apparatus main body 30 searches for min(X, Y) (step S119). Specifically, the acquisition function 384 sets the threshold TH acquired in step S109 to the distribution of the brightness values on the raster RA acquired in step S107, moves the point along the raster RA in the direction in which the depth decreases from the center position of the sample volume SV, and searches for a position where the brightness value of the point at each position is equal to or more than the threshold TH, thereby searching for min(X, Y).

A search method for min(X, Y) will be described with reference to FIGS. 10 and 11. As illustrated in FIG. 10, the acquisition function 384 sets the threshold acquired in step S109 to the distribution of the brightness value on the raster RA acquired in step S107. Then, as illustrated in FIG. 11, the acquisition function 384 moves the point along the raster RA in the direction in which the depth decreases from the coordinates (X_{c}, Y_{c}) of the center position of the sample volume, that is, the negative direction of the y-axis illustrated in FIG. 11, which is the opposite direction to the direction in which max(X, Y) is searched, and acquires the brightness value of the moved point. Then, the acquisition function 384 repeatedly moves the point along the raster RA in the negative direction of the y-axis illustrated in FIG. 11 to acquire the brightness value of each point, thereby searching for a point at which the brightness value is equal to or more than the threshold. Note that the processing in step S121 is equivalent to the processing in step S113 described above, and thus description thereof is omitted. Then, in step S121, when there is no point at which it is equal to or more than the threshold TH (step S121: No), the acquisition function 384 in the processing circuitry 38 of the apparatus main body 30 stores an error value in min(X, Y) (step S123).

On the other hand, in step S121, when there is a point at which it is equal to or more than the threshold TH (step S121: Yes), the acquisition function 384 in the processing circuitry 38 of the apparatus main body 30 stores min(X, Y) (step S125). Specifically, the acquisition function 384 stores, as min(X, Y), coordinates of a point moved back a predetermined value in the positive direction of the y-axis from a point at which the brightness value is equal to or more than the threshold TH. More specifically, for example, the acquisition function 384 stores, as min(X, Y), the coordinates of a point where the y coordinate is moved back only 1 from the coordinates of the first point at which the brightness value is equal to or more than the threshold TH along the raster RA.

Note that although the acquisition function 384 stores, as min(X, Y), the coordinates of a point where the y coordinate is moved back only 1 from the coordinates of a point at which the brightness value is equal to or more than the threshold TH, it is not limited thereto. That is, the coordinates stored as min(X, Y) by the acquisition function 384 are arbitrary, and for example, the acquisition function 384 may store, as min(X, Y), the coordinates of a point one or more points before the point at which the brightness value is equal to or more than the threshold TH, or may store, as min(X, Y), the coordinates of a point moved by a predetermined value from the point at which the brightness value is equal to or more than the threshold TH, based on the search result in step S11.

Next, as illustrated in FIG. 3, the acquisition function 384 acquires a blood vessel width candidate based on the B mode information (step S127). Specifically, the acquisition function 384 acquires the blood vessel width candidate based on the B mode information based on max(X, Y) and min(X, Y). More specifically, when no error value is stored in either max(X, Y) or min(X, Y), the acquisition function 384 acquires, for example, an absolute value of a difference between respective Y coordinates of max(X, Y) and min(X, Y) as the blood vessel width candidate. Further, when an error value is stored in at least one of max(X, Y) and min(X, Y), the acquisition function 384 acquires, for example, the error value as the blood vessel width candidate.

Then, in step S127, by acquiring the blood vessel width candidates based on the B mode information, the blood vessel width candidate acquiring processing based on the B mode information is ended.

Returning to FIG. 1, the adjustment function 385 adjusts the region included in the sample gate SG based on a plurality of blood vessel width candidates. For example, the adjustment function 385 adjusts the sample volume SV based on the plurality of blood vessel width candidates. In the present embodiment, the adjustment function 385 adjusts the width of the sample volume SV based on the plurality of blood vessel width candidates.

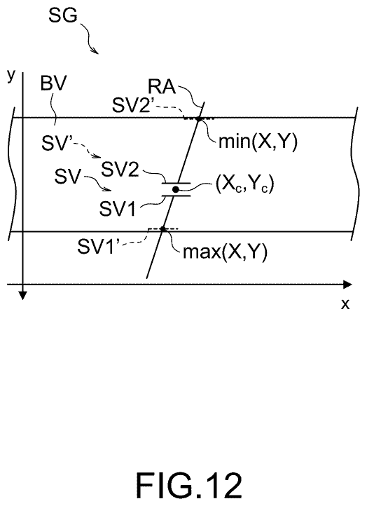

FIG. 12 is a diagram illustrating an example of a method of adjusting the sample volume SV. In the example illustrated in FIG. 12, the adjustment function 385 adjusts the width of the sample volume SV by moving a lower end SV1 of the sample volume SV to a position of max(X, Y) and moving an upper end of the sample volume SV to a position of min(X, Y) based on the plurality of blood vessel width candidates. As illustrated in FIG. 12, the adjusted sample volume SV′ is adjusted to a width corresponding to the blood vessel width of the blood vessel BV by a lower end SV1′ of the sample volume SV′ and an upper end SV2′ of the sample volume SV′ being located near the lower end and the upper end of the blood vessel BV.

The input device 50 includes a mouse, a keyboard, a button, a panel switch, a touch command screen, a wheel, a dial, a foot switch, a trackball, a joystick, and the like, receives various setting requests from the user of the ultrasonic diagnostic apparatus 1, and transfers the received various setting requests to the apparatus main body 30. The input device 50 includes, for example, a sample volume width adjustment button for starting adjustment of the sample volume SV.

The display 70 displays a graphical user interface (GUI) for the user of the ultrasonic diagnostic apparatus 1 to input various setting requests using the input device 50, and displays the ultrasonic image IM, the Doppler waveform, and the like generated in the apparatus main body 30. In addition, the display 70 displays various messages in order to notify the user of a processing status of the apparatus main body 30. Furthermore, the display 70 includes a speaker and can also output sound. For example, the speaker of the display 70 outputs a predetermined sound such as a beep sound in order to notify the user of the processing status of the apparatus main body 30. The display 70 corresponds to a display unit according to the present embodiment.

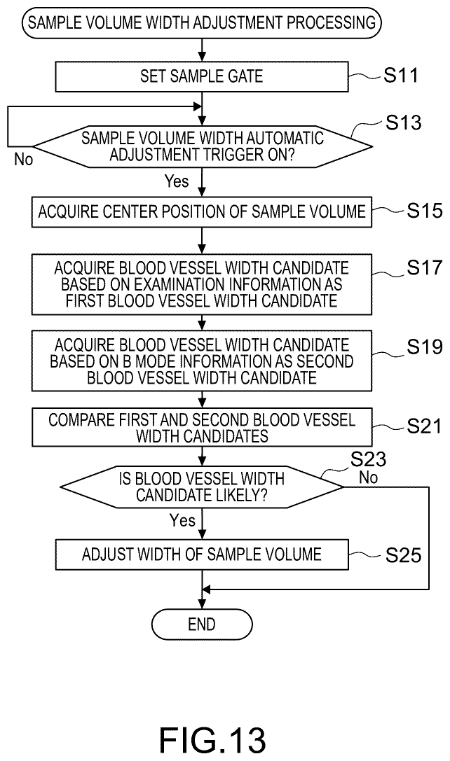

FIG. 13 is a flowchart for describing sample volume width adjustment processing executed in the ultrasonic diagnostic apparatus 1 according to the first embodiment; In the sample volume width adjustment processing illustrated in FIG. 13, the sample gate SG is set, the center position of the sample volume SV is acquired, the blood vessel width candidate is acquired based on the examination information, the blood vessel width candidate is acquired based on the B mode information, the blood vessel width candidates are compared, the likelihood of the blood vessel width is determined, and the width of the sample volume SV is adjusted. For example, the sample volume width adjustment processing is executed when the PWD mode is activated.

As illustrated in FIG. 13, first, the sample gate setting function 383 in the processing circuitry 38 of the apparatus main body 30 sets the sample gate SG (step S11). Specifically, the sample gate setting function 383 sets the sample gate SG including the sample volume SV in the B mode image IM1 which is the ultrasonic image IM in which the long axis image of the blood vessel BV is captured. More specifically, for example, the sample gate setting function 383 receives an input operation of setting the sample gate SG from the user, and sets the sample volume SV included in the sample gate SG at the position of the blood vessel BV depicted in the B mode image IM1 based on the received input operation of setting the sample gate SG.

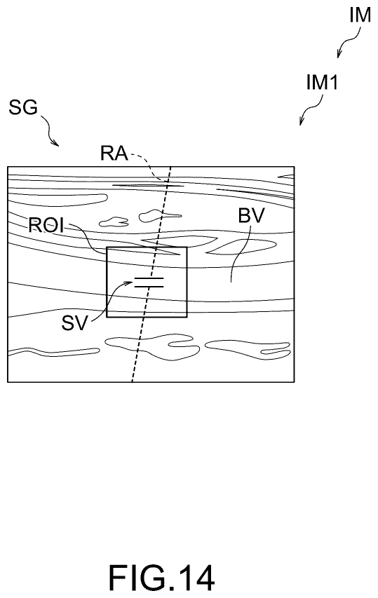

FIG. 14 is a diagram illustrating an example of the ultrasonic image IM in which the sample gate SG is set. As illustrated in FIG. 14, the sample gate SG is set in the B mode image IM1, which is the ultrasonic image IM, in which the long axis image of the blood vessel BV is captured. In the example illustrated in FIG. 14, the sample volume SV included in the sample gate SG is set on the blood vessel BV depicted on the B mode image IM1. In the example illustrated in FIG. 14, a region of interest ROI is set in the B mode image IM1 which is the ultrasonic image IM.

Next, as illustrated in FIG. 13, the sample gate setting function 383 determines whether or not a sample volume width automatic adjustment trigger is turned on (step S13). Specifically, the sample gate setting function 383 determines whether or not the sample volume width automatic adjustment trigger is turned on by determining whether or not the first input operation related to the adjustment of the width of the sample volume SV is received from the user. More specifically, after setting the sample gate SG to the ultrasonic image IM during the execution of the PWD mode scan, the sample gate setting function 383 determines whether or not pressing of the sample volume width adjustment button, which is a first input operation related to the received adjustment of the width of the sample volume SV, has been received, thereby determining whether or not the sample volume width automatic adjustment trigger has been turned on. Then, in step S13, when the sample volume width automatic adjustment trigger is not turned on (step S13: No), the processing of step S13 is repeated and the processing waits until the sample volume width automatic adjustment trigger is turned on.

On the other hand, when the sample volume width automatic adjustment trigger is turned on in step S13, that is, when the first input operation is received after the sample gate SG is set to the ultrasonic image IM during the execution of the PWD mode scan (step S13: Yes), the sample gate setting function 383 acquires the center position of the sample volume SV (step S15). Specifically, the sample gate setting function 383 acquires coordinates of the center position of the sample volume SV in the ultrasonic image IM. In the present embodiment, as illustrated in FIG. 13, when the region of interest ROI is set, the sample gate setting function 383 acquires the coordinates of the center position of the sample volume SV in the region of interest ROI.

Next, as illustrated in FIG. 13, the acquisition function 384 in the processing circuitry 38 of the apparatus main body 30 acquires a blood vessel width candidate based on the examination information as a first blood vessel width candidate (step S17). Specifically, the acquisition function 384 acquires the examination information and acquires the blood vessel width candidate based on the examination information, thereby acquiring the blood vessel width candidate based on the examination information as the first blood vessel width candidate. In the present embodiment, the acquisition function 384 acquires the carotid artery as the setting content of the examination mode included in the examination information, and acquires 5.4±1.0 mm as the blood vessel diameter of the carotid artery from the first relation table stored in the storage circuitry 37. The blood vessel width candidate based on the examination information is set as the maximum search range.

Next, as illustrated in FIG. 13, the acquisition function 384 acquires a blood vessel width candidate based on the B mode information as a second blood vessel width candidate (step S19). Specifically, the acquisition function 384 acquires the blood vessel width candidate based on the B mode information as the second blood vessel width candidate by executing the blood vessel width candidate acquiring processing based on the B mode information described above.

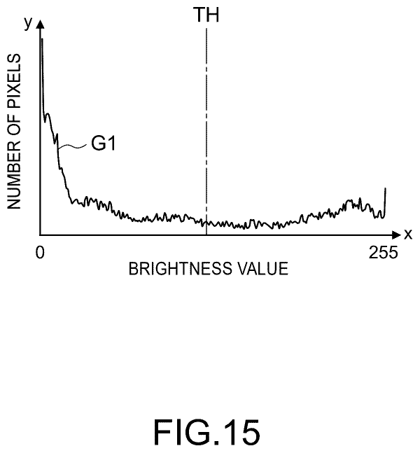

FIG. 15 is a diagram illustrating an example of distribution of brightness values in a certain range from the center position of the sample volume SV acquired in the ultrasonic image IM illustrated in FIG. 14, and is a diagram corresponding to FIG. 5. In the example illustrated in FIG. 15, the acquisition function 384 acquires a graph G1 indicating the distribution of the brightness values in the region of interest ROI illustrated in FIG. 14 as the distribution of the brightness values in a certain range from the center position of the sample volume SV. In addition, the acquisition function 384 acquires the threshold TH based on the distribution of the brightness values of the region of interest ROI illustrated in FIG. 15.

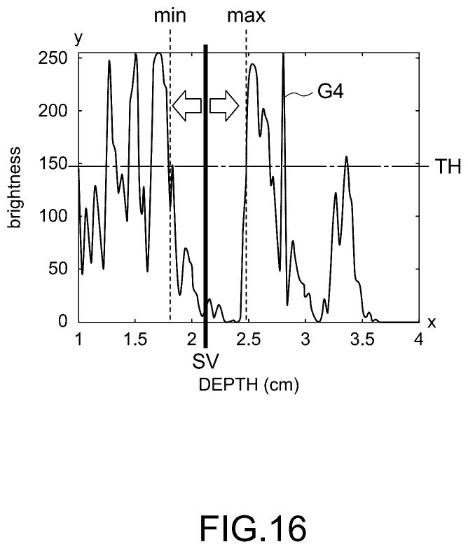

FIG. 16 is a diagram illustrating an example of distribution of brightness values on the raster RA acquired in the ultrasonic image IM illustrated in FIG. 14, and is a diagram corresponding to FIG. 7. In the example illustrated in FIG. 16, the acquisition function 384 acquires a graph G4 in which the x-axis is Depth and the y-axis is a brightness value as the distribution of the brightness value on the raster RA in the sample gate SG of the ultrasonic image IM illustrated in FIG. 14. Then, as illustrated in FIG. 16, the acquisition function 384 sets the threshold TH acquired from the distribution of brightness values in a certain range from the center position of the sample volume SV illustrated in FIG. 15 in the graph G4, searches for max(X, Y) and min(X, Y), and stores max(X, Y) and min(X, Y). In the example illustrated in FIG. 16, the acquisition function 384 acquires 5.2 mm as the blood vessel width candidate based on the B mode information.

Next, as illustrated in FIG. 13, the adjustment function 385 in the processing circuitry 38 of the apparatus main body 30 compares the first blood vessel width candidate with the second blood vessel width candidate (step S21). Specifically, the adjustment function 385 compares, based on the plurality of blood vessel width candidates, a plurality of blood vessel width candidates. More specifically, based on the blood vessel width candidate based on the examination information acquired as the first blood vessel width candidate in step S17 and the blood vessel width candidate based on the B mode information acquired as the second blood vessel width candidate in step S19, the adjustment function 385 compares the blood vessel width candidate based on the examination information acquired as the first blood vessel width candidate in step S17 with the blood vessel width candidate based on the B mode information acquired as the second blood vessel width candidate in step S19. For example, the adjustment function 385 compares whether the blood vessel width candidate based on the B mode information is within the range of the blood vessel width candidate based on the examination information.

Next, as illustrated in FIG. 13, the adjustment function 385 determines whether or not the blood vessel width candidate is likely (step S23). Specifically, the adjustment function 385 determines whether or not the blood vessel width candidate is likely based on a comparison result that is a result of comparing the plurality of blood vessel width candidates. More specifically, the adjustment function 385 determines whether or not the blood vessel width candidate based on the B mode information is likely based on a comparison result between the blood vessel width candidate based on the examination information acquired as the first blood vessel width candidate and the blood vessel width candidate based on the B mode information acquired as the second blood vessel width candidate. For example, when at least one of the first blood vessel width candidate and the second blood vessel width candidate has an error value, the adjustment function 385 determines that the blood vessel width candidate is not likely. For example, when the blood vessel width candidate based on the B mode information acquired as the second blood vessel width candidate is not within the range of the blood vessel width candidate based on the acquired examination information as the first blood vessel width candidate, the adjustment function 385 determines that the blood vessel width candidate is not likely. On the other hand, for example, when the blood vessel width candidate based on the B mode information is within the range of the blood vessel width candidate based on the examination information, the adjustment function 385 determines that the blood vessel width candidate is likely. In the present embodiment, when the range of the blood vessel width candidate based on the examination information acquired in step S17 is 5.4±1.0 mm and the blood vessel width candidate based on the B mode information acquired in step S19 is 5.2 mm, the blood vessel width candidate based on the B mode information is within the range of the blood vessel width candidate based on the examination information, and thus the adjustment function 385 determines that the blood vessel width candidate is likely.

Then, when it is determined in step S23 that the blood vessel width candidate is likely (step S23: Yes), the adjustment function 385 adjusts the width of the sample volume SV (step S25). Specifically, when it is determined that the blood vessel width candidate is likely, the adjustment function 385 adjusts the width of the sample volume SV based on the blood vessel width candidate determined to be likely. More specifically, when it is determined that the blood vessel width candidate based on the B mode information is likely, the adjustment function 385 adjusts the width of the sample volume SV based on the blood vessel width candidate based on the B mode information.

In the present embodiment, for example, the adjustment function 385 adjusts the width of the sample volume SV by using the following Expressions (1) and (2) based on max(X, Y) and min(X, Y) in the blood vessel width candidate based on the B mode information acquired in step S19, the width of the sample volume SV before adjustment included in the sample gate SG set in step S11, and the center position of the sample volume SV acquired in step S15.

$$\begin{matrix}[ Math . 1 ] & \\ Extension rate of upper end = \frac{( {Y}_{c} - {Y}_{min} )}{W / 2} & ( 1 )\end{matrix}$$

$$\begin{matrix}[ Math . 2 ] & \\ Extension rate of lower end = \frac{( {Y}_{max} - {Y}_{c} )}{W / 2} & ( 2 )\end{matrix}$$

W is the width of the sample volume SV before adjustment, that is, the width of the sample volume SV included in the information regarding the setting of the sample gate, Yₘᵢₙ is the y coordinate of min(X, Y), and Yₘₐₓ is the y coordinate of max(X, Y).

The adjustment function 385 adjusts the upper end SV2 of the sample volume SV by moving the position of the upper end SV2 of the sample volume SV along the raster RA so as to extend the distance in the y-axis direction from the center position of the sample volume SV to the upper end SV2 of the sample volume SV according to an extension rate of the upper end SV2 of the sample volume SV calculated from the above-described Expression (1). In addition, the adjustment function 385 adjusts the lower end SV1 of the sample volume SV by moving the position of the lower end SV1 of the sample volume SV along the raster RA so as to extend the distance in the y-axis direction from the center position of the sample volume SV to the lower end SV1 of the sample volume SV according to the extension rate of the lower end SV1 of the sample volume SV calculated from the above-described Expression (2). Then, the width of the sample volume SV is adjusted by adjusting each of the upper end SV2 and the lower end SV1 of the sample volume SV.

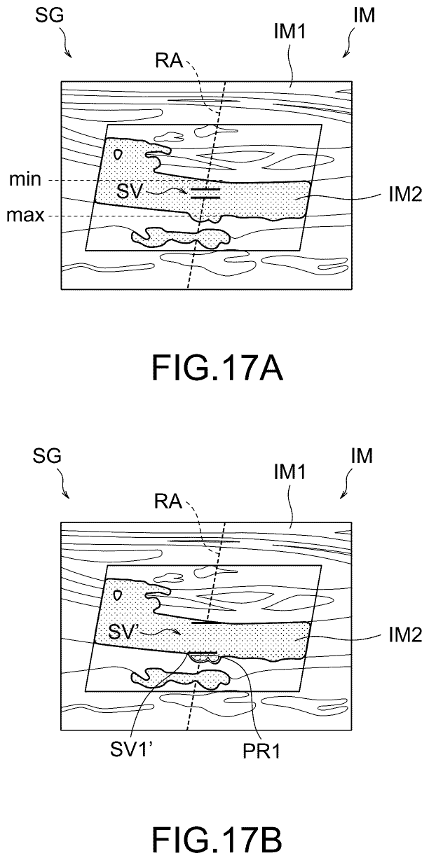

FIGS. 17A and 17B are diagrams illustrating an example of the ultrasonic image IM in which the sample gate SG including the sample volume SV before and after adjustment is set. As illustrated in FIG. 17A, before adjustment by the adjustment function 385, the sample volume SV does not have an appropriate width with respect to the blood vessel width. On the other hand, as illustrated in FIG. 17B, after the adjustment by the adjustment function 385, the sample volume SV′ has an appropriate width with respect to the blood vessel width. In addition, as illustrated in FIG. 17B, on the lower end side of the sample volume SV′, a protruding region PR1 of the blood vessel BV is generated in a color Doppler image IM2 superimposed on the B mode image IM1, but the lower end SV1′ of the sample volume is adjusted without being affected by the protruding region PR1.

In step S23, when the blood vessel width candidate is not likely (step S23: No), or by adjusting the width of the sample volume SV in step S25, the sample volume width adjustment processing ends.

As described above, in the ultrasonic diagnostic apparatus 1, the sample gate SG including the sample volume SV is set in the B mode image IM1 which is the ultrasonic image IM, the blood vessel width candidate based on the examination information and the blood vessel width candidate based on the B mode information are acquired as the plurality of blood vessel width candidates for the blood vessel at the position where the sample gate SG is set, and the sample volume SV included in the sample gate SG is adjusted based on the blood vessel width candidate based on the examination information and the blood vessel width candidate based on the B mode information. Therefore, it is not necessary to manually adjust the sample volume SV for each blood vessel, and the sample volume SV can be appropriately adjusted according to the blood vessel width, so that the examination efficiency and the examination accuracy in the PWD mode can be improved.

In addition, in the ultrasonic diagnostic apparatus 1, the sample volume SV included in the sample gate SG is adjusted based on the blood vessel width candidate based on the examination information and the blood vessel width candidate based on the B mode information, and thus it is possible to determine the blood vessel width with high accuracy by adjusting the sample volume SV using the B mode information with high resolution.

### Second Embodiment

In the ultrasonic diagnostic apparatus 1 according to the first embodiment described above, the adjustment function 385 compares the first blood vessel width candidate with the second blood vessel width candidate to determine the likelihood of the blood vessel width, and does not adjust the sample volume SV when it is determined that the blood vessel width candidate is not likely, but it is not limited thereto. In a second embodiment, the ultrasonic diagnostic apparatus 1 that acquires a third blood vessel width candidate when it is determined that the blood vessel width candidate is not likely will be described.

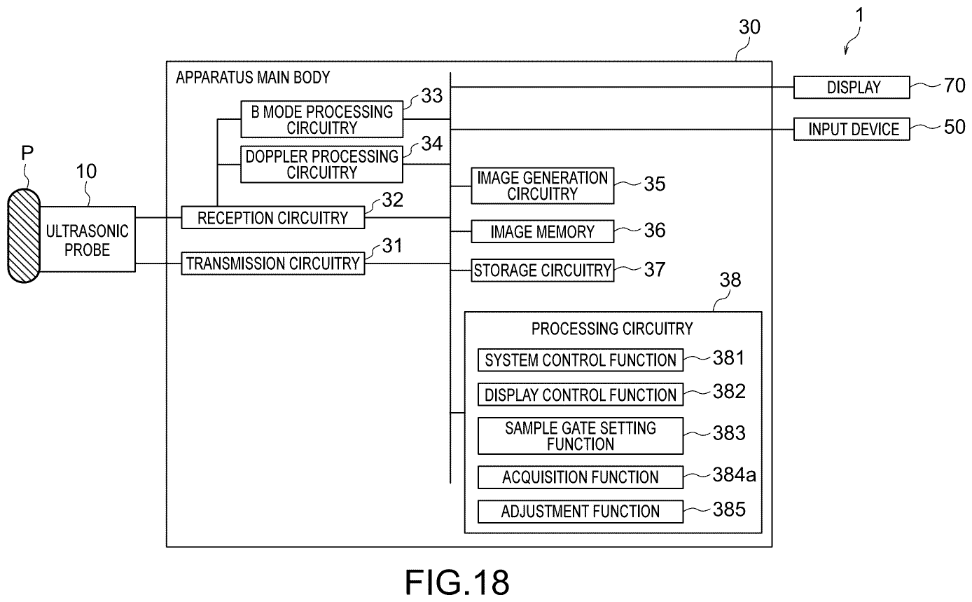

FIG. 18 is a block diagram illustrating an example of a configuration of the ultrasonic diagnostic apparatus 1 according to the second embodiment, and is a diagram corresponding to FIG. 1. As illustrated in FIG. 18, in the ultrasonic diagnostic apparatus 1 according to the second embodiment, the acquisition function of the processing circuitry 38 is different from that of the first embodiment described above, and thus is denoted as an acquisition function 384a. Note that configurations and functions other than the acquisition function 384a are the same as those in FIG. 1, and thus description thereof will be omitted.

The acquisition function 384a acquires a third blood vessel width candidate when the blood vessel width candidate is not likely. Specifically, the acquisition function 384a acquires a blood vessel width candidate as the third blood vessel width candidate based on the power information. The blood vessel width candidate acquired based on the power information is, for example, a blood vessel width candidate acquired based on a power value estimated at each of a plurality of sample points in Doppler data that is raw data before the scan conversion processing or a power image for display after the scan conversion processing. In particular, the blood vessel width candidate acquired based on the power value estimated at each of the plurality of sample points in the Doppler data, which is the raw data before the scan conversion processing, is the blood vessel width candidate acquired based on the data before being converted into the brightness value by the scan conversion by the image generation circuitry 35, and thus the acquisition function 384a can acquire the blood vessel width candidate with higher accuracy by acquiring the blood vessel width candidate acquired based on the power value estimated at each of the plurality of sample points in the Doppler data, which is the raw data before the scan conversion processing. Note that the blood vessel width candidate acquired based on the power information may be a blood vessel width candidate acquired based on information regarding the power in the Doppler data before the MTI filter is applied. In the following description, a case where the blood vessel width candidate acquired based on the power information is a blood vessel width candidate acquired based on the power information including the power value estimated at each of the plurality of sample points in the Doppler data that is the raw data before the scan conversion processing will be described as an example.

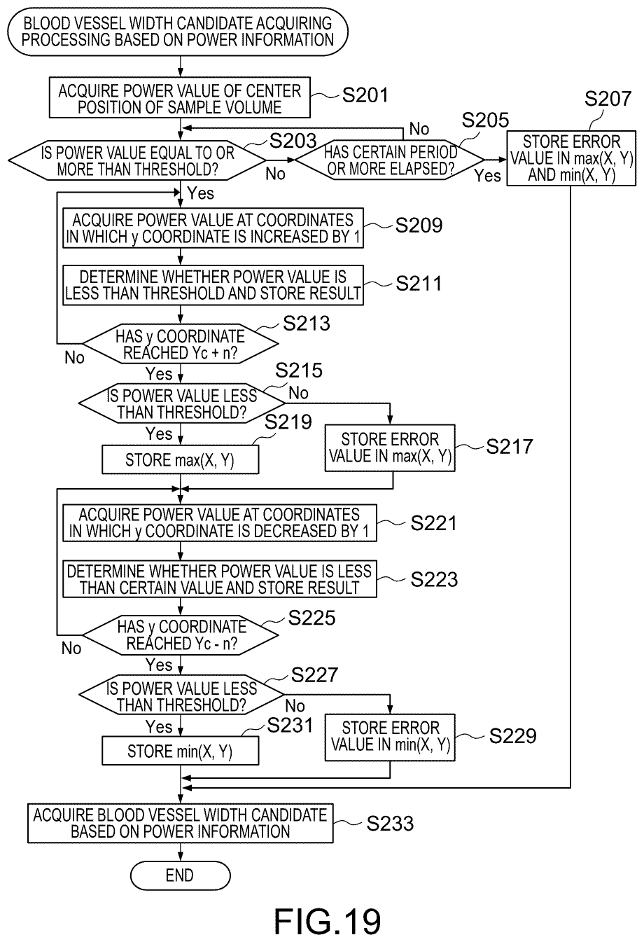

FIG. 19 is a flowchart for describing the blood vessel width candidate acquiring processing based on the power information executed in the apparatus main body 30. In the blood vessel width candidate acquiring processing based on the power information, a power value at the center position of the sample volume SV is acquired using the power information, a power value on the raster RA is acquired, whether or not the power value is less than a predetermined value is determined, max(X, Y) and min(X, Y) are stored, and a blood vessel width candidate based on the power information is acquired.

As illustrated in FIG. 19, first, the acquisition function 384a in the processing circuitry 38 of the apparatus main body 30 acquires the power value at the center position of the sample volume SV (step S201). Specifically, the acquisition function 384a acquires the power value of the blood flow at the center position of the sample volume SV included in the sample gate SG set in the color Doppler image IM2 using the color Doppler mode.

Next, as illustrated in FIG. 19, the acquisition function 384a determines whether or not the power value is equal to or more than a threshold (step S203). Specifically, the acquisition function 384a determines whether or not the power value of the blood flow at the center position of the sample volume SV acquired in step S201 is equal to or more than the threshold. As the threshold in step S203, for example, the threshold used when the clutter signal is suppressed in the MTI filter is used.

Then, in step S203, when the power value is not equal to or more than the threshold, that is, when the power value is less than the threshold (step S203: No), the acquisition function 384a determines whether or not a certain period or more has elapsed (step S205). Specifically, the acquisition function 384a determines whether or not a certain period or more has elapsed since the start of the blood vessel width candidate acquiring processing based on the power information. The certain period is, for example, a period set by the user such as 1 second for 60 heartbeats.

Then, in step S205, in a case where the certain period or more has not elapsed (step S205: No), the processing from steps S201 to S205 is repeated until the power value becomes equal to or more than the threshold or the certain period or more has elapsed. That is, in a case where the power value is less than the threshold, it is recognized that there is pulsatility in the blood flow, and a time phase in which the power value of the blood flow equal to or more than the threshold is generated is waited.

On the other hand, when the certain period or more has elapsed in step S205 (step S205: No), the acquisition function 384 stores error values in max(X, Y) and min(X, Y) (step S207).

On the other hand, in step S203, in a case where the power value is equal to or more than the threshold (step S203: Yes), the acquisition function 384a acquires the power value at the coordinates in which the y coordinate is increased by 1 (step S209). Specifically, the acquisition function 384a acquires the power value of the blood flow at the position of the coordinates in which the y coordinate is increased by 1 along the raster RA. Note that, in step S209, the acquisition function 384a acquires the power value of the blood flow at the position of the coordinates in which the y coordinate is increased by 1, but the value added to the y coordinate is not limited to 1. That is, the value added to the y coordinate is arbitrary, and may be equal to or more than 0 and less than 1, or may be more than 1.

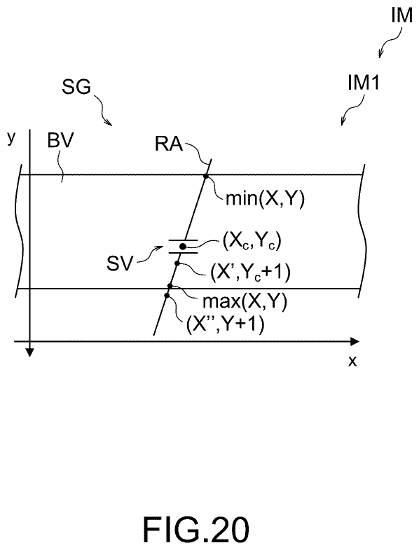

FIG. 20 is a diagram illustrating an example of a method of acquiring a power value. In the example illustrated in FIG. 20, the ultrasonic image IM is the color Doppler image IM2 superimposed on the B mode image IM1. In addition, the sample gate SG is set in the color Doppler image IM2 superimposed on the B mode image IM1 which is the ultrasonic image IM, and the sample volume SV included in the sample gate SG is arranged at the coordinates (X_{c}, Y_{c}) of the center position. In the example illustrated in FIG. 20, in step S209, the acquisition function 384a acquires the power value of the blood flow at the position of the coordinates (X′, Y_{c}+1) in which the y coordinate of the coordinates (X_{c}, Y_{c}) of the center position is increased by 1 along the raster RA.

Next, as illustrated in FIG. 19, the acquisition function 384a determines whether the power value is less than the threshold and stores the result (step S211). Specifically, the acquisition function 384a determines whether or not the power value of the blood flow acquired in step S209 is less than the threshold, and stores the coordinates of the position where the power value of the blood flow is acquired and the determination result in the storage circuitry 37 in association with each other.

Next, as illustrated in FIG. 19, the acquisition function 384a determines whether or not the y coordinate has reached Y_{c}+n (step S213). Specifically, the acquisition function 384a determines whether or not the y coordinate at the time of acquiring the power value in step S209 has reached Y_{c}+n. For example, in a case where the value of n is set to 9, the value of n indicates that the acquisition of the power value of the blood flow at Y_{c}+9, that is, the position of the coordinates in which the y coordinate is increased by 1 is repeated nine times. Then, the accuracy of the blood vessel determination increases as the value of n increases, that is, the accuracy of the blood vessel determination depends on the value of n. The value of n may be arbitrarily set by the user, or a fixed value may be set. Note that, in step S213, the acquisition function 384a determines whether or not the y coordinate has reached Y_{c}+n, but it is not limited thereto. For example, the acquisition function 384a may determine whether or not the y coordinate has reached a value of the y coordinate arbitrarily set by the user, or may determine whether or not the y coordinate has reached a lower end of the scan range in the ultrasonic image IM.

Then, in step S213, when the y coordinate has not reached Y_{c}+n (step S213: No), the acquisition function 384a returns to step S209, repeats the processing of acquiring the power value (step S209), the processing of determining whether the power value is less than the threshold and storing the result (step S211), and the processing of determining whether or not the y coordinate has reached Y_{c}+n (step S213), and waits until the y coordinate reaches Y_{c}+n.

On the other hand, in step S213, when the y coordinate reaches Y_{C}+n (step S213: Yes), the acquisition function 384a determines whether or not the power value is less than the threshold (step S215). Specifically, the acquisition function 384a determines whether or not the power value is less than the threshold based on the determination result stored in step S211. Then, in step S215, when the power value is not less than the threshold (step S215: No), the acquisition function 384a stores an error value in max(X, Y) (step S217).

On the other hand, in step S215, when the power value is less than the threshold (step S215: Yes), the acquisition function 384a stores max(X, Y) (step S219). Specifically, the acquisition function 384a stores, as max(X, Y), coordinates of a position when the determination result is first less than the threshold among the determination results in which the power value stored in step S211 is less than the threshold, that is, coordinates obtained by moving back a predetermined value in the negative direction of the y-axis from the coordinates in which the value of the y coordinate of the position where the power value of the blood flow corresponding to the determination result in which the power value is less than the threshold is acquired is smallest. More specifically, along the raster RA, the acquisition function 384a stores, as max(X, Y), coordinates obtained by moving back only 1 in the negative direction of the y-axis from the coordinates in which the value of the y coordinate of the position where the power value of the blood flow corresponding to the determination result in which the power value is less than the threshold is acquired is smallest. For example, in the example illustrated in FIG. 20, since the coordinates in which the value of the y coordinate at the position where the power value of the blood flow corresponding to the determination result in which the power value is less than the threshold is acquired is smallest is (X″, Y+1), the acquisition function 384a stores (X′, Y) in which the y coordinate is moved back only 1 from (X″, Y+1) along the raster RA as max(X, Y).

After the processing of steps S217 and S219, as illustrated in FIG. 19, the acquisition function 384a acquires the power value at the coordinates in which the y coordinate is decreased by 1 (step S221). Specifically, the acquisition function 384a acquires the power value of the blood flow at the position of the coordinates in which the y coordinate is decreased by 1 along the raster RA. More specifically, for example, in the example illustrated in FIG. 20, in step S221, the acquisition function 384a acquires the power value of the blood flow at the position of the coordinates (X′, Y_{c}−1) in which the y coordinate of the coordinates (X_{c}, Y_{c}) of the center position is decreased by 1 along the raster RA. Note that, in step S209, the acquisition function 384a acquires the power value of the blood flow at the position of the coordinates in which the y coordinate is decreased by 1, but the value subtracted from the y coordinate is not limited to 1. That is, the value subtracted from the y coordinate is arbitrary, and may be equal to or more than 0 and less than 1, or may be more than 1.

Next, as illustrated in FIG. 19, the acquisition function 384a in the processing circuitry 38 of the apparatus main body 30 determines whether the power value is less than the threshold, and stores the result (step S223). Specifically, the acquisition function 384a determines whether or not the power value of the blood flow acquired in step S221 is less than the threshold, and stores the coordinates of the position where the power value of the blood flow is acquired and the determination result in the storage circuitry 37 in association with each other.

Next, as illustrated in FIG. 19, the acquisition function 384a determines whether or not the y coordinate has reached Y_{c}−n (step S225). Specifically, the acquisition function 384a determines whether or not the y coordinate at the time of acquiring the power value in step S221 has reached Y_{c}-n. For example, when the value of n is set to 9, the value of n indicates that the acquisition of the power value of the blood flow at the position of Y_{c}-9, that is, the coordinates in which the y coordinate is decreased by 1 is repeated nine times. Then, the accuracy of the blood vessel determination increases as the value of n increases, that is, the accuracy of the blood vessel determination depends on the value of n. The value of n may be arbitrarily set by the user, or a fixed value may be set. Note that, in step S221, the acquisition function 384a determines whether or not the y coordinate has reached Y_{c}−n, but it is not limited thereto. For example, the acquisition function 384a may determine whether or not the y coordinate has reached a value of the y coordinate arbitrarily set by the user, or may determine whether or not the y coordinate has reached an upper end of the scan range in the ultrasonic image IM.

Then, in step S225, when the y coordinate has not reached Y_{c}−n (step S225: No), the acquisition function 384a returns to step S221, repeats the processing of acquiring the power value (step S221), the processing of determining whether the power value is less than the threshold and storing the result (step S223), and the processing of determining whether or not the y coordinate has reached Y_{c}−n (step S225), and waits until the y coordinate reaches Y_{c}−n.

On the other hand, in step S225, when the y coordinate reaches Y_{c}−n (step S225: Yes), the acquisition function 384a determines whether or not the power value is less than the threshold (step S227). Specifically, the acquisition function 384a determines whether or not the power value is less than the threshold based on the determination result stored in step S223. Then, in step S227, when the power value is not less than the threshold (step S227: No), the acquisition function 384a stores an error value in min(X, Y) (step S229).

On the other hand, in step S227, when the power value is less than the threshold (step S227: Yes), the acquisition function 384a stores min(X, Y) (step S231). Specifically, the acquisition function 384a stores, as min(X, Y), coordinates of a position when the determination result is first less than the threshold among the determination results in which the power value stored in step S223 is less than the threshold, that is, coordinates obtained by moving back a predetermined value in the positive direction of the y-axis from the coordinates in which the value of the y coordinate of the position where the power value of the blood flow corresponding to the determination result in which the power value is less than the threshold is acquired is largest. More specifically, along the raster RA, the acquisition function 384a stores, as min(X, Y), coordinates obtained by moving back only 1 in the positive direction of the y-axis from the coordinates in which the value of the y coordinate of the position where the power value of the blood flow corresponding to the determination result in which the power value is less than the threshold is acquired is largest.

Next, as illustrated in FIG. 19, the acquisition function 384a acquires a blood vessel width candidate based on the power information (step S233). Specifically, the acquisition function 384a acquires the blood vessel width candidate based on the power information based on max(X, Y) and min(X, Y). More specifically, when no error value is stored in either max(X, Y) or min(X, Y), the acquisition function 384a acquires, for example, an absolute value of a difference between respective y coordinates of max(X, Y) and min(X, Y) as the blood vessel width candidate. Further, when an error value is stored in at least one of max(X, Y) and min(X, Y), the acquisition function 384a acquires, for example, the error value as a blood vessel width candidate.

Then, in step S233, by acquiring the blood vessel width candidates based on the power information, the blood vessel width candidate acquiring processing based on the power information is ended.

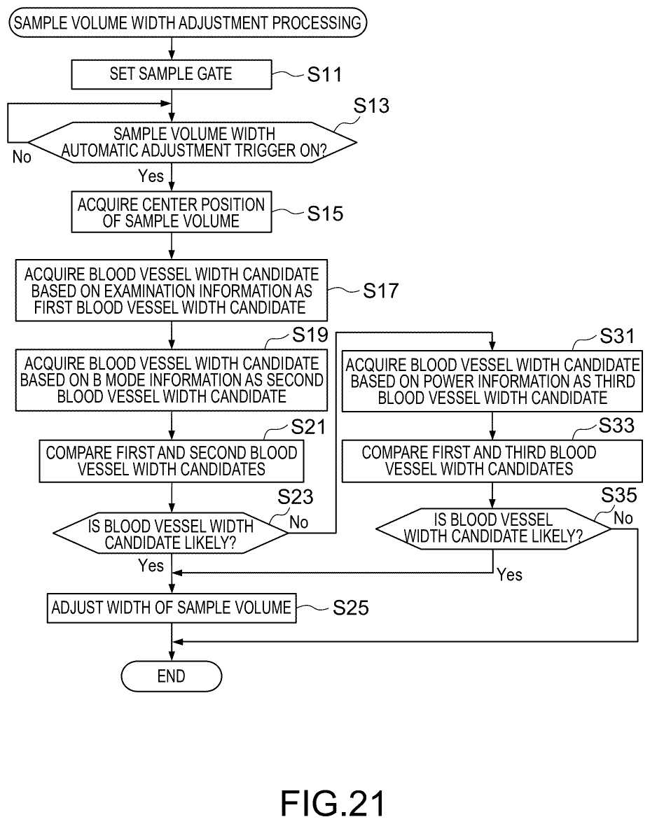

FIG. 21 is a flowchart for describing the sample volume width adjustment processing executed in the ultrasonic diagnostic apparatus 1 according to the second embodiment, and is a diagram corresponding to FIG. 13. In the sample volume width adjustment processing illustrated in FIG. 21, when the blood vessel width candidate is not likely, the blood vessel width candidate based on the power information is acquired, the blood vessel width candidate based on the examination information is compared with the blood vessel width candidate based on the power information, the likelihood of the blood vessel width is determined, and the width of the sample volume SV is adjusted. For example, the sample volume width adjustment processing is executed when the PWD mode is activated. Note that the processing in steps S11 to S17 illustrated in FIG. 21 is the same as that in FIG. 13, and thus the description thereof will be omitted.

Next, as illustrated in FIG. 21, the acquisition function 384a in the processing circuitry 38 of the apparatus main body 30 acquires a blood vessel width candidate as a second blood vessel width candidate based on the B mode information (step S19a). Specifically, the acquisition function 384a acquires the blood vessel width candidate based on the B mode information as the second blood vessel width candidate by executing the blood vessel width candidate acquiring processing based on the B mode information described above.

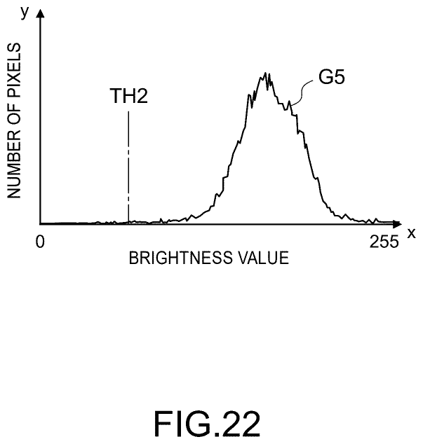

FIG. 22 is a diagram illustrating an example of distribution of brightness values in a certain range from the center position of the sample volume SV acquired in the ultrasonic image IM, and is a diagram corresponding to FIG. 15. In the example illustrated in FIG. 22, the acquisition function 384a acquires a graph G5 indicating the distribution of the brightness values in the region of interest ROI set on the ultrasonic image IM as the distribution of the brightness values in a certain range from the center position of the sample volume SV. In addition, the acquisition function 384a acquires the threshold TH2 based on the distribution of the brightness values of the region of interest ROI illustrated in FIG. 22.

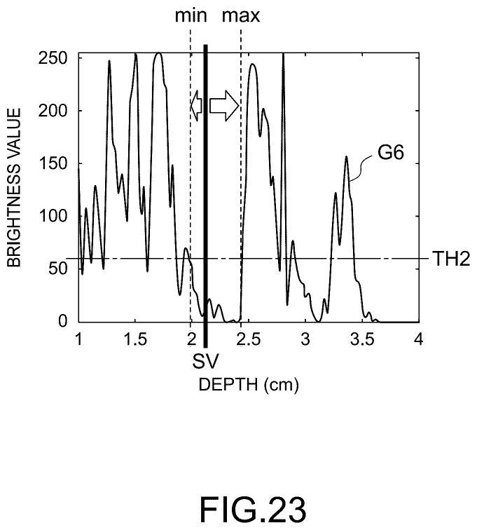

FIG. 23 is a diagram illustrating an example of distribution of brightness values on the raster RA acquired in the ultrasonic image IM illustrated in FIG. 22, and is a diagram corresponding to FIG. 16. In the example illustrated in FIG. 23, the acquisition function 384a acquires a graph G6 in which the x-axis is Depth and the y-axis is a brightness value as the distribution of the brightness value on the raster RA in the sample gate SG of the ultrasonic image IM. Then, as illustrated in FIG. 23, the acquisition function 384a sets the threshold TH2 acquired from the distribution of brightness values in a certain range from the center position of the sample volume SV of FIG. 22 in the graph G6, searches for max(X, Y) and min(X, Y), and stores max(X, Y) and min(X, Y). In the example illustrated in FIG. 23, the acquisition function 384a acquires 3.1 mm as the blood vessel width candidate based on the B mode information. Note that processing in step S21 is the same as that in FIG. 13, and thus the description thereof will be omitted.

Next, as illustrated in FIG. 21, the adjustment function 385 in the processing circuitry 38 of the apparatus main body 30 determines whether or not the blood vessel width candidate is likely (step S23a). In the present embodiment, when the range of the blood vessel width candidate based on the examination information acquired in step S17 is 5.4±1.0 mm, whereas the blood vessel width candidate based on the B mode information acquired in step S19a is 3.1 mm, the adjustment function 385 determines that the blood vessel width candidate is not likely because the blood vessel width candidate based on the B mode information is not within the range of the blood vessel width candidate based on the examination information. Note that the description of step S23a other than that described above is equivalent to the description of step S23 in FIG. 13, and thus the description thereof will be omitted.

When it is determined in step S23a that the blood vessel width candidate is not likely (step S23a: No), the acquisition function 384a acquires the blood vessel width candidate based on the power information as the third blood vessel width candidate (step S31). Specifically, the acquisition function 384a acquires the blood vessel width candidate based on the power information as the third blood vessel width candidate by executing the blood vessel width candidate acquiring processing based on the power information described above.

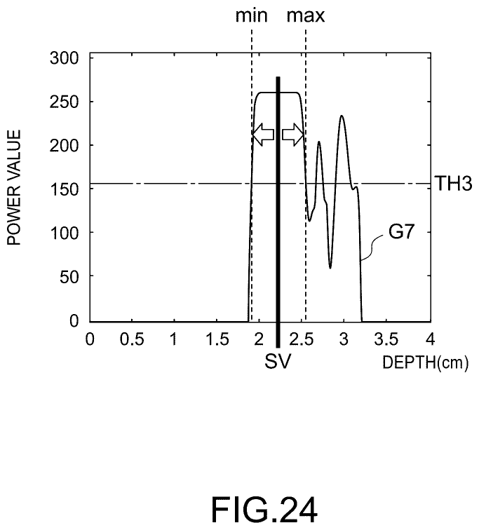

FIG. 24 is a diagram illustrating an example of a distribution of power values on the raster RA. In the example illustrated in FIG. 24, the acquisition function 384a acquires a graph G7 in which the x-axis is Depth and the y-axis is the power value as the distribution of the power value on the raster RA. The graph G7 is acquired by executing the processing from step S209 to step S213 and the processing from step S221 to step S225 in the blood vessel width candidate acquiring processing based on the power information. Further, as illustrated in FIG. 24, the threshold TH3 used in step S211 and step S223 in the blood vessel width candidate acquiring processing based on the power information is set in the distribution of the power value on the raster RA. In the example illustrated in FIG. 24, the acquisition function 384a acquires 6.2 mm as the blood vessel width candidate based on the power information.

Next, as illustrated in FIG. 21, the adjustment function 385 compares the first blood vessel width candidate with the third blood vessel width candidate (step S33). Specifically, the adjustment function 385 compares, based on the plurality of blood vessel width candidates, a plurality of blood vessel width candidates. More specifically, the adjustment function 385 compares the blood vessel width candidate based on the examination information acquired as the first blood vessel width candidate in step S17 with the blood vessel width candidate based on the power information acquired as the third blood vessel width candidate in step S31. For example, the adjustment function 385 compares whether the blood vessel width candidate based on the power information is within the range of the blood vessel width candidate based on the examination information.

Next, as illustrated in FIG. 21, the adjustment function 385 determines whether or not the blood vessel width candidate is likely (step S35). Specifically, the adjustment function 385 determines whether or not the blood vessel width candidate is likely based on a comparison result that is a result of comparing the plurality of blood vessel width candidates. More specifically, the adjustment function 385 determines whether or not the blood vessel width candidate based on the power information is likely based on a comparison result between the blood vessel width candidate based on the examination information acquired as the first blood vessel width candidate and the blood vessel width candidate based on the power information acquired as the third blood vessel width candidate. For example, when at least one of the first blood vessel width candidate and the third blood vessel width candidate has an error value, it is determined that the blood vessel width candidate is not likely. Furthermore, for example, when the blood vessel width candidate based on the power information is not within the range of the blood vessel width candidate based on the examination information, the adjustment function 385 determines that it is not likely. On the other hand, for example, when the blood vessel width candidate based on the power information is within the range of the blood vessel width candidate based on the examination information, the adjustment function 385 determines that the blood vessel width candidate is likely. In the present embodiment, for example, when the range of the blood vessel width candidate based on the examination information acquired in step S17 is 5.4±1.0 mm and the blood vessel width candidate based on the power information acquired in step S31 is 6.2 mm, the blood vessel width candidate based on the power information is within the range of the blood vessel width candidate based on the examination information, and thus the adjustment function 385 determines that the blood vessel width candidate is likely.

Then, when it is determined in step S35 that the blood vessel width candidate is likely (step S35: Yes), the adjustment function 385 adjusts the width of the sample volume SV (step S25a). Specifically, the adjustment function 385 adjusts the width of the sample volume SV based on the blood vessel width candidate based on the power information determined to be within the range of the blood vessel width candidate based on the examination information in step S23a.

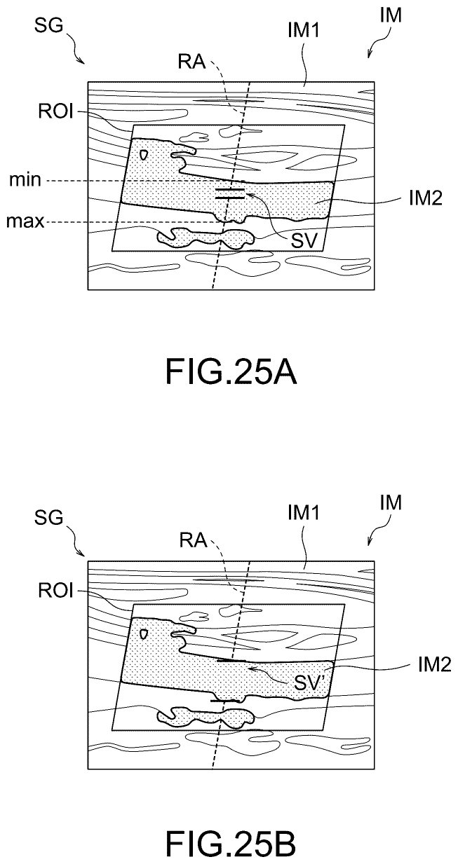

FIGS. 25A and 25B are diagrams illustrating an example of the ultrasonic image IM in which the sample gate SG including the sample volume SV before and after adjustment of the width of the sample volume SV is set. As illustrated in FIG. 25A, before adjustment by the adjustment function 385, the sample volume SV does not have an appropriate width with respect to the blood vessel width. On the other hand, as illustrated in FIG. 25B, after the adjustment by the adjustment function 385, the sample volume SV′ has an appropriate width with respect to the blood vessel width.

In step S35, when the blood vessel width candidate is not likely (step S35: No), or by adjusting the width of the sample volume SV in step S25a, the sample volume width adjustment processing ends.

As described above, in the ultrasonic diagnostic apparatus 1, the sample gate SG including the sample volume SV is set in the B mode image IM1 which is the ultrasonic image IM, the blood vessel width candidate based on the examination information and the blood vessel width candidate based on the B mode information are acquired as the plurality of blood vessel width candidates for the blood vessel at the position where the sample gate SG is set, and when the blood vessel width candidate is not likely, the blood vessel width candidate based on the power information is acquired, and the sample volume SV included in the sample gate SG is adjusted based on the blood vessel width candidate based on the examination information and the blood vessel width candidate based on the power information. Therefore, it is not necessary to manually adjust the sample volume SV for each blood vessel, and the sample volume SV can be appropriately adjusted according to the blood vessel width, so that the examination efficiency and the examination accuracy in the PWD mode can be improved.

### Third Embodiment

In the ultrasonic diagnostic apparatus 1 according to the first and second embodiments described above, the width of the sample volume is not adjusted when the blood vessel width candidate is not likely by comparing the blood vessel width candidate based on the examination information with the blood vessel width candidate other than the blood vessel width candidate based on the examination information acquired by the acquisition function, but the method of adjusting the width of the sample volume is not limited thereto. In a third embodiment, the width of the sample volume SV may be adjusted based on one of max(X, Y) and min(X, Y) in the blood vessel width candidate based on the B mode information and the blood vessel width candidate based on the examination information even when the blood vessel width candidate is not likely by comparing the blood vessel width candidate based on the examination information with the blood vessel width candidate other than the blood vessel width candidate based on the examination information acquired by the acquisition function. A case in which this modification is applied to the above-described second embodiment will be referred to as the third embodiment, and portions different from those of the above-described second embodiment will be described. Note that, in the following, a case in which this modification is applied to the first embodiment will be described, but this modification is also applicable to the second embodiment described above.

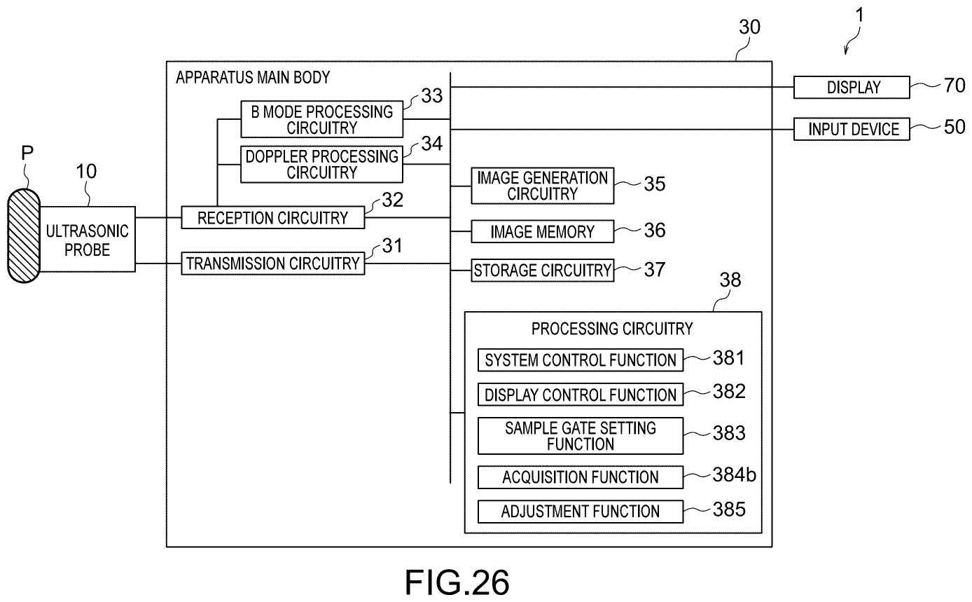

FIG. 26 is a block diagram illustrating an example of a configuration of the ultrasonic diagnostic apparatus 1 according to the third embodiment, and is a diagram corresponding to FIG. 1. As illustrated in FIG. 26, in the ultrasonic diagnostic apparatus 1 according to the third embodiment, the acquisition function of the processing circuitry 38 is different from that of the first embodiment described above, and thus is denoted as an acquisition function 384b. Note that configurations and functions other than the acquisition function 384b are the same as those in FIG. 1, and thus description thereof will be omitted.

The acquisition function 384b acquires a blood vessel width candidate based on the velocity information as a fourth blood vessel width candidate. In addition, the acquisition function 384b acquires a blood vessel width candidate based on the dispersion information as a fifth blood vessel width candidate. The blood vessel width candidate acquired based on the velocity information is, for example, a blood vessel width candidate acquired based on a velocity value estimated at each of a plurality of sample points in Doppler data that is raw data before the scan conversion processing or a velocity image for display after the scan conversion processing. In addition, the blood vessel width candidate acquired based on the dispersion information is, for example, a blood vessel width candidate acquired based on a dispersion value estimated at each of a plurality of sample points in Doppler data that is raw data before the scan conversion processing or a velocity image for display after the scan conversion processing. In particular, the blood vessel width candidate acquired based on the velocity value or the dispersion value estimated at each of the plurality of sample points in the Doppler data, which is the raw data before the scan conversion processing, is the blood vessel width candidate acquired based on the data before being converted into the brightness value by the scan conversion by the image generation circuitry 35, and thus the acquisition function 384b can acquire the blood vessel width candidate with higher accuracy by acquiring the blood vessel width candidate acquired based on the velocity value or the dispersion value estimated at each of the plurality of sample points in the Doppler data, which is the raw data before the scan conversion processing. Note that the blood vessel width candidate acquired based on the velocity information or the dispersion information may be a blood vessel width candidate acquired based on information regarding the velocity or information regarding the dispersion in the Doppler data before the MTI filter is applied. In the following description, a case where the blood vessel width candidate acquired based on the velocity information is a blood vessel width candidate acquired based on velocity information or dispersion information including a velocity value or a dispersion value estimated at each of a plurality of sample points in Doppler data that is raw data before the scan conversion processing will be described as an example.

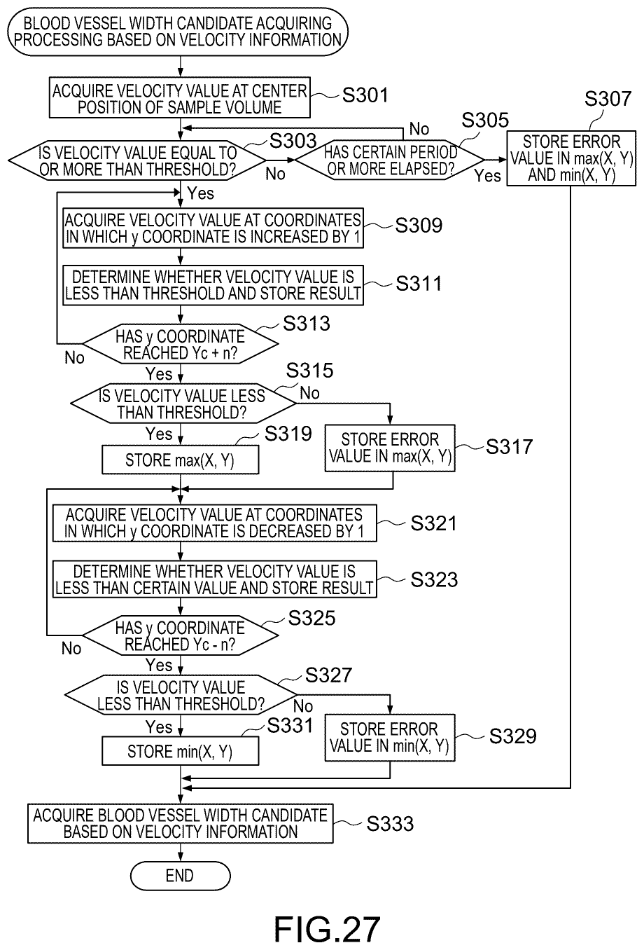

FIG. 27 is a flowchart for describing the blood vessel width candidate acquiring processing based on velocity information executed in the apparatus main body 30. In the blood vessel width candidate acquiring processing based on the velocity information, a velocity value at the center position of the sample volume SV is acquired using the velocity information, a velocity value on the raster RA is acquired, whether or not the velocity value is less than a predetermined value is determined, max(X, Y) and min(X, Y) are stored, and a blood vessel width candidate based on the velocity information is acquired.

As illustrated in FIG. 27, first, the acquisition function 384b in the processing circuitry 38 of the apparatus main body 30 acquires the velocity value at the center position of the sample volume SV (step S301). Specifically, the acquisition function 384b acquires the velocity value of the blood flow at the center position of the sample volume SV included in the sample gate SG set in the color Doppler image IM2 using the color Doppler mode.

Next, as illustrated in FIG. 27, the acquisition function 384b determines whether or not the velocity value is equal to or more than a threshold (step S303). Specifically, the acquisition function 384b determines whether or not the velocity value of the blood flow at the center position of the sample volume SV acquired in step S301 is equal to or more than the threshold. As the threshold in step S303, for example, the threshold used when the clutter signal is suppressed in the MTI filter is used.

Then, in step S303, when the velocity value is not equal to or more than the threshold, that is, when the velocity value is less than the threshold (step S303: No), the acquisition function 384b determines whether or not a certain period or more has elapsed (step S305). Specifically, the acquisition function 384b determines whether or not a certain period or more has elapsed since the start of the blood vessel width candidate acquiring processing based on the velocity information. The certain period is, for example, a period set by the user such as 1 second for 60 heartbeats.

Then, in step S305, in a case where the certain period or more has not elapsed (step S305: No), the processing from steps S301 to S305 is repeated until the velocity value becomes equal to or more than the threshold or the certain period or more has elapsed. That is, in a case where the velocity value is less than the threshold, it is recognized that there is pulsatility in the blood flow, and a time phase when the velocity value of the blood flow equal to or more than the threshold occurs is waited.

On the other hand, when the certain period or more has elapsed in step S305 (step S305: No), the acquisition function 384 stores error values in max(X, Y) and min(X, Y) (step S307).

On the other hand, in step S303, in a case where the velocity value is equal to or more than the threshold (step S303: Yes), the acquisition function 384b acquires the velocity value at the coordinates in which the y coordinate is increased by 1 (step S309). Specifically, the acquisition function 384b acquires the velocity value of the blood flow at the position of the coordinates in which the y coordinate is increased by 1 along the raster RA. More specifically, for example, in the example illustrated in FIG. 20, in step S309, the acquisition function 384b acquires the velocity value of the blood flow at the position of the coordinates (X′, Y_{c}+1) in which the y coordinate of the coordinates (X_{c}, Y_{c}) of the center position is increased by 1 along the raster RA. Note that, in step S309, the acquisition function 384b acquires the velocity value of the blood flow at the position of the coordinates in which the y coordinate is increased by 1, but the value added to the y coordinate is not limited to 1. That is, the value added to the y coordinate is arbitrary, and may be equal to or more than 0 and less than 1, or may be more than 1.

Next, as illustrated in FIG. 27, the acquisition function 384b determines whether the velocity value is less than the threshold and stores the result (step S311). Specifically, the acquisition function 384b determines whether or not the velocity value of the blood flow acquired in step S309 is less than the threshold, and stores the coordinates of the position where the velocity value of the blood flow is acquired and the determination result in the storage circuitry 37 in association with each other.

Next, as illustrated in FIG. 27, the acquisition function 384b determines whether or not the y coordinate has reached Y_{c}+n (step S313). Specifically, the acquisition function 384b determines whether or not the y coordinate at the time of acquiring the velocity value in step S309 has reached Y_{c}+n. For example, when the value of n is set to 9, the value of n indicates that the acquisition of the velocity value of the blood flow at the position of Y_{c}+9, that is, the position of the coordinates in which the y coordinate is increased by 1 is repeated nine times. Then, the accuracy of the blood vessel determination increases as the value of n increases, that is, the accuracy of the blood vessel determination depends on the value of n. The value of n may be arbitrarily set by the user, or a fixed value may be set. Note that, in step S313, the acquisition function 384b determines whether or not the y coordinate has reached Y_{c}+n, but it is not limited thereto. For example, the acquisition function 384b may determine whether or not the y coordinate has reached a value of the y coordinate arbitrarily set by the user, or may determine whether or not the y coordinate has reached the lower end of the scan range in the ultrasonic image IM.

Then, in step S313, when the y coordinate has not reached Y_{c}+n (step S313: No), the acquisition function 384b returns to step S309, repeats the processing of acquiring the velocity value (step S309), the processing of determining whether the velocity value is less than the threshold and storing the result (step S311), and the processing of determining whether or not the y coordinate has reached Y_{c}+n (step S313), and waits until the y coordinate reaches Y_{c}+n.

On the other hand, when the y coordinate reaches Y_{c}+n in step S313 (step S313: Yes), the acquisition function 384b determines whether or not the velocity value is less than the threshold (step S315). Specifically, the acquisition function 384b determines whether or not the velocity value is less than the threshold based on the determination result stored in step S311. Then, in step S315, when the velocity value is not less than the threshold (step S315: No), the acquisition function 384b stores an error value in max(X, Y) (step S317).

On the other hand, when the velocity value is less than the threshold in step S315 (step S315: Yes), the acquisition function 384b stores max(X, Y) (step S319). Specifically, the acquisition function 384b stores, as max(X, Y), coordinates of a position when the determination result is first less than the threshold among the determination results in which the velocity value stored in step S311 is less than the threshold, that is, coordinates obtained by moving back a predetermined value in the negative direction of the y-axis from the coordinates in which the value of the y coordinate of the position where the velocity value of the blood flow corresponding to the determination result in which the velocity value is less than the threshold is acquired is smallest. More specifically, along the raster RA, the acquisition function 384b stores, as max(X, Y), coordinates obtained by moving back only 1 in the negative direction of the y-axis from the coordinates in which the value of the y coordinate of the position where the velocity value of the blood flow corresponding to the determination result in which the velocity value is less than the threshold is acquired is smallest. For example, in the example illustrated in FIG. 20, since the coordinates in which the value of the y coordinate at the position where the velocity value of the blood flow corresponding to the determination result in which the velocity value is less than the threshold is acquired is smallest is (X″, Y+1), the acquisition function 384b stores (X′, Y) in which the y coordinate is moved back only 1 from (X″, Y+1) along the raster RA as max(X, Y).

After the processing of steps S317 and S319, as illustrated in FIG. 27, the acquisition function 384b acquires the velocity value at the coordinates in which the y coordinate is decreased by 1 (step S321). Specifically, the acquisition function 384b acquires the velocity value of the blood flow at a position of coordinates in which the y coordinate of the center position of the sample volume SV is decreased by 1 along the raster RA. More specifically, for example, in step S321, the acquisition function 384a acquires the power value of the blood flow at the position of the coordinates (X′, Y_{c}−1) in which the y coordinate of the coordinates (X_{c}, Y_{c}) of the center position of the sample volume SV is decreased by 1 along the raster RA. Note that, in step S309, the acquisition function 384b acquires the velocity value of the blood flow at the position of the coordinates in which the y coordinate is decreased by 1, but the value subtracted from the y coordinate is not limited to 1. That is, the value subtracted from the y coordinate is arbitrary, and may be equal to or more than 0 and less than 1, or may be more than 1. Note that the processing in step S323 after step S321 is the same as the processing in step S311, and thus description thereof will be omitted.

Next, as illustrated in FIG. 27, the acquisition function 384b determines whether or not the y coordinate has reached Y_{c}−n (step S325). Specifically, the acquisition function 384b determines whether or not the y coordinate at the time of acquiring the velocity value in step S321 has reached Y_{c}−n. For example, when the value of n is set to 9, the value of n indicates that the acquisition of the velocity value of the blood flow at the position of Y_{c}−9, that is, the coordinates in which the y coordinate is decreased by 1 is repeated nine times. Then, the accuracy of the blood vessel determination increases as the value of n increases, that is, the accuracy of the blood vessel determination depends on the value of n. The value of n may be arbitrarily set by the user, or a fixed value may be set. Note that, in step S321, the acquisition function 384b determines whether or not the y coordinate has reached Y_{c}−n, but it is not limited thereto. For example, the acquisition function 384b may determine whether or not the y coordinate has reached a value of the y coordinate arbitrarily set by the user, or may determine whether or not the y coordinate has reached the upper end of the scan range in the ultrasonic image IM.

Then, in step S325, when the y coordinate has not reached Y_{c}−n (step S325: No), the acquisition function 384b returns to step S321, repeats the processing of acquiring the velocity value (step S321), the processing of determining whether the velocity value is less than the threshold and storing the result (step S323), and the processing of determining whether or not the y coordinate has reached Y_{c}−n (step S325), and waits until the y coordinate reaches Y_{c}−n.

On the other hand, when the y coordinate reaches Y_{c}−n in step S325 (step S325: Yes), the acquisition function 384b determines whether or not the velocity value is less than the threshold (step S327). Specifically, the acquisition function 384b determines whether or not the velocity value is less than the threshold based on the determination result stored in step S323. Then, when the velocity value is not less than the threshold in step S327 (step S327: No), the acquisition function 384b in the processing circuitry 38 of the apparatus main body 30 stores the error value in min(X, Y) (step S329).

On the other hand, when the velocity value is less than the threshold in step S327 (step S327: Yes), the acquisition function 384b in the processing circuitry 38 of the apparatus main body 30 stores min(X, Y) (step S331). Specifically, the acquisition function 384b stores, as min(X, Y), coordinates of a position when the determination result is first less than the threshold among the determination results in which the velocity value stored in step S323 is less than the threshold, that is, coordinates obtained by moving back a predetermined value in the positive direction of the y-axis from the coordinates where the value of the y coordinate of the position where the velocity value of the blood flow corresponding to the determination result in which the velocity value is less than the threshold is acquired is largest. More specifically, along the raster RA, the acquisition function 384b stores, as min(X, Y), coordinates obtained by moving back only 1 in the positive direction of the y-axis from the coordinates in which the value of the y coordinate of the position where the velocity value of the blood flow corresponding to the determination result in which the velocity value is less than the threshold is acquired is largest.

Next, as illustrated in FIG. 27, the acquisition function 384b acquires a blood vessel width candidate based on the velocity information (step S333). Specifically, the acquisition function 384b acquires a blood vessel width candidate based on the velocity information based on max(X, Y) and min(X, Y). More specifically, when no error value is stored in either max(X, Y) or min(X, Y), the acquisition function 384b acquires, for example, an absolute value of a difference between respective y coordinates of max(X, Y) and min(X, Y) as the blood vessel width candidate. Further, when an error value is stored in at least one of max(X, Y) and min(X, Y), the acquisition function 384b acquires, for example, the error value as the blood vessel width candidate.

Then, in step S333, the blood vessel width candidate based on the velocity information is acquired, and the blood vessel width candidate acquiring processing based on the velocity information is ended.

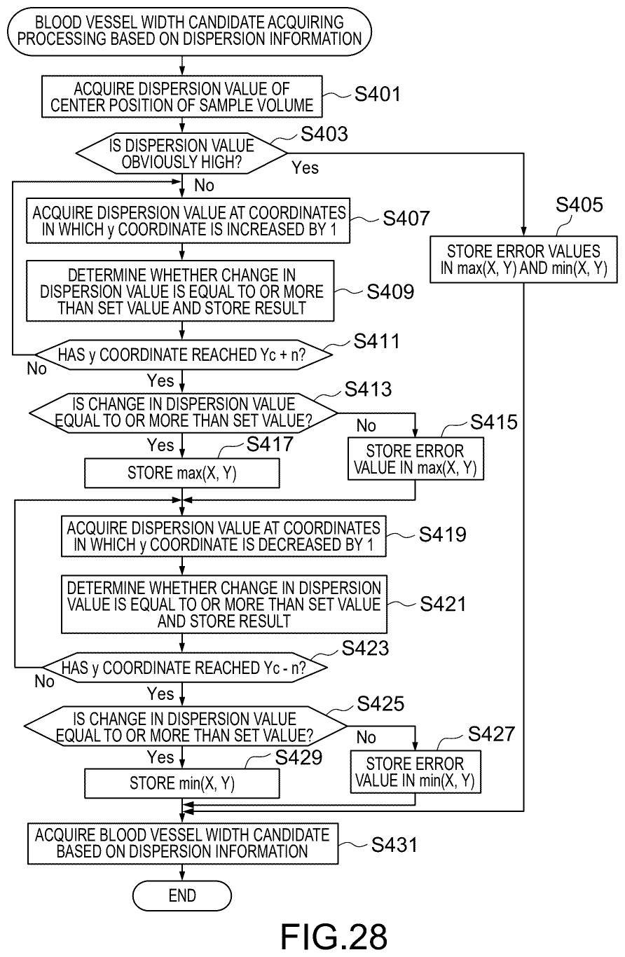

FIG. 28 is a flowchart for describing the blood vessel width candidate acquiring processing based on dispersion information executed in the apparatus main body 30. In the blood vessel width candidate acquiring processing based on the dispersion information, a dispersion value of the center position of the sample volume is acquired using the dispersion information, a dispersion on the raster RA is acquired, whether or not the dispersion is equal to or more than a threshold is determined, max(X, Y) and min(X, Y) are stored, and a blood vessel width candidate based on the dispersion information is acquired.

As illustrated in FIG. 28, first, the acquisition function 384b in the processing circuitry 38 of the apparatus main body 30 acquires the dispersion value of the center position of the sample volume SV (step S401). Specifically, the acquisition function 384b acquires the dispersion value of the blood flow at the center position of the sample volume SV acquired by the sample gate setting function 383 using the color Doppler mode. More specifically, in the example illustrated in FIG. 20, the acquisition function 384b acquires the dispersion value of the coordinates (X_{c}, Y_{c}) of the center position of the sample volume SV.

Next, as illustrated in FIG. 28, the acquisition function 384b determines whether or not the dispersion value is obviously high (step S403). Specifically, the acquisition function 384b determines whether or not the dispersion value of the blood flow at the center position of the sample volume SV acquired in step S301 is obviously high by determining whether or not the dispersion value is equal to or more than a threshold. Then, in step S403, when the dispersion value is obviously high (step S403: Yes), the acquisition function 384b stores error values in max(X, Y) and min(X, Y) (step S405).

On the other hand, in step S403, when the dispersion value is not obviously high (step S403: No), the acquisition function 384b acquires the dispersion value at the coordinates in which the y coordinate is increased by 1 (step S407). Specifically, the acquisition function 384b acquires the dispersion value of the blood flow at the position of the coordinates in which the y coordinate is increased by 1 along the raster RA. More specifically, for example, in the example illustrated in FIG. 20, in step S407, the acquisition function 384b acquires the velocity value of the blood flow at the position of the coordinates (X′, Y_{c}+1) in which the y coordinate is increased by 1 from the center position of the sample volume SV along the raster RA. Note that, in step S407, the acquisition function 384b acquires the dispersion value of the blood flow at the position of the coordinates in which the y coordinate is increased by 1, but the value added to the y coordinate is not limited to 1. That is, the value added to the y coordinate is arbitrary, and may be equal to or more than 0 and less than 1, or may be more than 1.

Next, as illustrated in FIG. 27, the acquisition function 384b determines whether a change in the dispersion value is equal to or more than a set value and stores the result (step S409). Specifically, the acquisition function 384b determines whether or not a change in the dispersion value of the blood flow is equal to or more than the set value, and stores the dispersion value, the coordinates of the position where the dispersion value of the blood flow is acquired, and the determination result in the storage circuitry 37 in association with each other. More specifically, the acquisition function 384b compares the dispersion value acquired in step S407 with the dispersion value acquired immediately before the dispersion value, and determines whether or not the change in the dispersion value is equal to or more than the set value by determining whether the dispersion significantly increases. For example, in a case where the acquisition function 384b compares a dispersion value s²(X′, Y_{c}+1) at the position of (X′, Y_{c}+1) with a dispersion value s²(X_{c}, Y_{c}) at the position of (X_{c}, Y_{c}) immediately before (X′, Y_{c}+1) and determines whether the dispersion significantly increases, the acquisition function determines whether or not the change in the dispersion value is equal to or more than the set value depending on whether or not the following Expression (3) is satisfied.

$$\begin{matrix}[ Math . 3 ] & \\ \frac{{s}^{2} ( {X}^{′} , {Y}_{c} + 1 ) - {s}^{2} ( {X}_{c} , {Y}_{c} )}{{s}^{2} ( {X}_{c} , {Y}_{c} )} \geq Set value & ( 3 )\end{matrix}$$

Next, as illustrated in FIG. 27, the acquisition function 384b determines whether or not the y coordinate has reached Y_{c}+n (step S411). Specifically, the acquisition function 384b determines whether or not the y coordinate at the time of acquiring the dispersion value in step S407 has reached Y_{c}+n. For example, when the value of n is set to 9, the value of n indicates that the acquisition of the dispersion value of the blood flow at the position of Y_{c}+9, that is, the coordinates in which the y coordinate is increased by 1 is repeated nine times. Then, the accuracy of the blood vessel determination increases as the value of n increases, that is, the accuracy of the blood vessel determination depends on the value of n. The value of n may be arbitrarily set by the user, or a fixed value may be set. Note that, in step S411, the acquisition function 384b determines whether or not the y coordinate has reached Y_{c}+n, but it is not limited thereto. For example, the acquisition function 384b may determine whether or not the y coordinate has reached a value of the y coordinate arbitrarily set by the user, or may determine whether or not the y coordinate has reached the lower end of the scan range in the ultrasonic image IM.

Then, in step S411, when the y coordinate has not reached Y_{c}+n (step S411: No), the acquisition function 384b returns to step S407, repeats the processing of acquiring the dispersion value (step S407), the processing of determining whether the change in the dispersion value is equal to or more than the set value and storing the result (step S409), and the processing of determining whether or not the y coordinate has reached Y_{c}+n (step S411), and waits until the y coordinate reaches Y_{c}+n.

On the other hand, in step S411, when the y coordinate reaches Y_{c}+n (step S411: Yes), the acquisition function 384b determines whether or not the change in the dispersion value is equal to or more than the set value (step S413). Specifically, the acquisition function 384b determines whether or not the change in the dispersion value is equal to or more than the set value based on the determination result stored in step S311. Then, in step S413, when the change in the dispersion value is not equal to or larger than the set value (step S413: No), the acquisition function 384b stores an error value in max(X, Y) (step S415).

On the other hand, in step S413, when the change in the dispersion value is equal to or more than the set value (step S413: Yes), the acquisition function 384b stores max(X, Y) (step S417). Specifically, the acquisition function 384b stores, as max(X, Y), coordinates of a position when the determination result first becomes equal to or more than the set value among the determination results, which are stored in step S409, in which the change in the dispersion value is equal to or more than the set value, that is, coordinates obtained by moving back a predetermined value in the negative direction of the y-axis from the coordinates in which the value of the y coordinate of the position where the dispersion value of the blood flow corresponding to the determination result in which the change is equal to or more than the set value is acquired is smallest. More specifically, along the raster RA, the acquisition function 384b stores, as max(X, Y), coordinates obtained by moving back only 1 in the negative direction of the y-axis from the coordinates in which the value of the y coordinate of the position where the dispersion value of the blood flow corresponding to the determination result in which the change in the dispersion value is equal to or more than the set value is acquired is smallest. For example, in the example illustrated in FIG. 20, since the coordinates in which the value of the y coordinate at the position where the dispersion value of the blood flow corresponding to the determination result in which the change in the dispersion value is equal to or more than the set value is acquired is smallest are (X″, Y+1), the acquisition function 384b stores (X′, Y) in which the y coordinate is moved back only 1 from (X″, Y+1) along the raster RA as max(X, Y).

After the processing of steps S415 and S417, as illustrated in FIG. 28, the acquisition function 384b acquires the dispersion value at the coordinate in which the y coordinate is decreased by 1 (step S419). Specifically, the acquisition function 384b acquires the dispersion value of the blood flow at a position of coordinates in which the y coordinate of the center position of the sample volume SV is decreased by 1 along the raster RA. Note that, in step S309, the acquisition function 384b acquires the velocity value of the blood flow at the position of the coordinates in which the y coordinate is decreased by 1, but the value subtracted from the y coordinate is not limited to 1. That is, the value subtracted from the y coordinate is arbitrary, and may be equal to or more than 0 and less than 1, or may be more than 1. Note that the processing in step S421 after step S419 is the same as the processing in step S409, and thus description thereof will be omitted.

Next, as illustrated in FIG. 28, the acquisition function 384b determines whether or not the y coordinate has reached Y_{c}−n (step S423). Specifically, the acquisition function 384b determines whether or not the y coordinate at the time of acquiring the dispersion value in step S419 has reached Y_{c}−n. For example, when the value of n is set to 9, the value of n indicates that the acquisition of the dispersion value of the blood flow at the position of Y_{c}−9, that is, the coordinates in which the y coordinate is decreased by 1 is repeated nine times. Then, the accuracy of the blood vessel determination increases as the value of n increases, that is, the accuracy of the blood vessel determination depends on the value of n. The value of n may be arbitrarily set by the user, or a fixed value may be set. Note that, in step S321, the acquisition function 384b determines whether or not the y coordinate has reached Y_{c}−n, but it is not limited thereto. For example, the acquisition function 384b may determine whether or not the y coordinate has reached a value of the y coordinate arbitrarily set by the user, or may determine whether or not the y coordinate has reached the upper end of the scan range in the ultrasonic image IM.

Then, in step S423, when the y coordinate has not reached Y_{c}−n (step S423: No), the acquisition function 384b returns to step S407, repeats the processing of acquiring the dispersion value (step S419), the processing of determining whether the change in the dispersion value is equal to or more than the set value and storing the result (step S421), and the processing of determining whether or not the y coordinate has reached Y_{c}−n (step S423), and waits until the y coordinate reaches Y_{c}−n.

On the other hand, in step S423, when the y coordinate reaches Y_{c}−n (step S423: Yes), the acquisition function 384b determines whether or not the change in the dispersion value is equal to or more than the set value (step S425). Specifically, the acquisition function 384b determines whether or not the change in the dispersion value is equal to or more than the set value based on the determination result stored in step S311. Then, in step S425, when the change in the dispersion value is not equal to or more than the set value (step S425: No), the acquisition function 384b stores an error value in min(X, Y) (step S427).

On the other hand, in step S413, when the change in the dispersion value is equal to or more than the set value (step S425: Yes), the acquisition function 384b stores min(X, Y) (step S429). Specifically, the acquisition function 384b stores, as min(X, Y), coordinates of a position when the determination result first becomes equal to or more than the set value among the determination results in which the change value of the dispersion value stored in step S323 is equal to or more than the set value, that is, coordinates obtained by moving back a predetermined value in the positive direction of the y-axis from coordinates in which the value of the y coordinate of the position where the dispersion value of blood flow corresponding to the determination result in which the change of the dispersion value is equal to or more than the set value is acquired is largest. More specifically, along the raster RA, the acquisition function 384b stores, as min(X, Y), coordinates obtained by moving back only 1 in the positive direction of the y-axis from the coordinates in which the value of the y coordinate of the position where the dispersion value of the blood flow corresponding to the determination result in which the change in the dispersion value is equal to or more than the set value is acquired is largest.

Next, as illustrated in FIG. 28, the acquisition function 384b acquires a blood vessel width candidate based on the dispersion information (step S431). Specifically, the acquisition function 384b acquires the blood vessel width candidate based on the dispersion information based on max(X, Y) and min(X, Y). More specifically, when no error value is stored in either max(X, Y) or min(X, Y), the acquisition function 384b acquires, for example, an absolute value of a difference between respective y coordinates of max(X, Y) and min(X, Y) as the blood vessel width candidate. Further, when an error value is stored in at least one of max(X, Y) and min(X, Y), the acquisition function 384b acquires, for example, the error value as the blood vessel width candidate.

Then, in step S431, by acquiring the blood vessel width candidates based on the dispersion information, the blood vessel width candidate acquiring processing based on the dispersion information is ended.

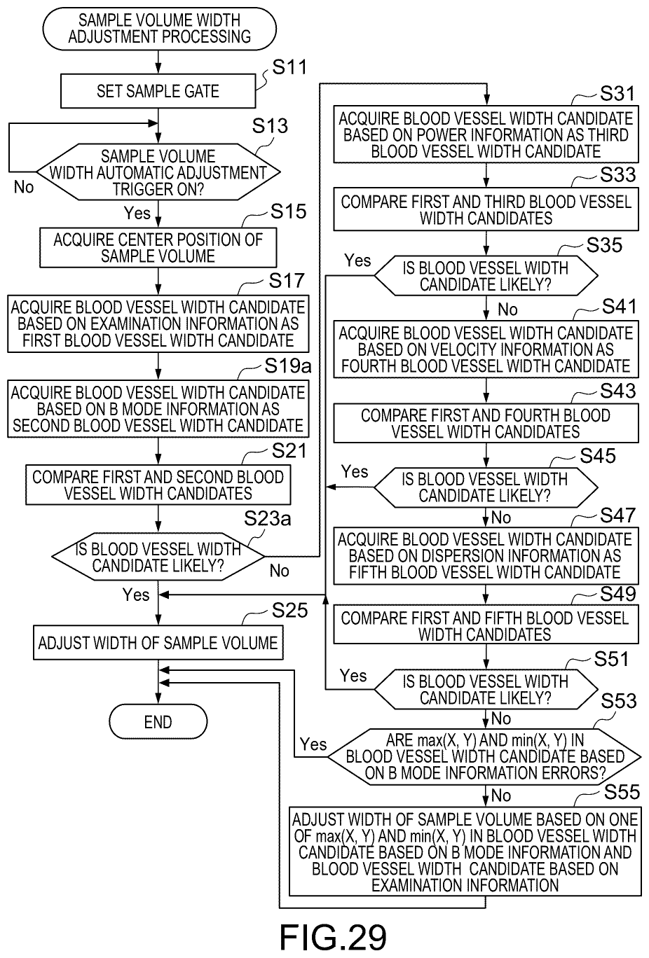

FIG. 29 is a flowchart for describing sample volume width adjustment processing executed in the ultrasonic diagnostic apparatus 1 according to the third embodiment; In the sample volume width adjustment processing illustrated in FIG. 29, when the blood vessel width candidate based on the power information is not likely, a blood vessel width candidate based on the velocity information is acquired, a blood vessel width candidate based on the examination information is compared with the blood vessel width candidate based on the velocity information, when the blood vessel width candidate based on the velocity information is not likely, a blood vessel width candidate based on the dispersion information is acquired, the blood vessel width candidate based on the examination information is compared with a blood vessel width candidate based on the dispersion information, or when the blood vessel width candidate based on the dispersion information is not likely, the width of the sample volume SV is adjusted by applying the blood vessel width candidate of the examination information to the position of the blood vessel wall that is likely to be the B mode information. For example, the sample volume width adjustment processing is executed when the PWD mode is activated. Note that processing in steps S11 to S17 and S21 illustrated in FIG. 29 is the same as that in FIG. 13, and thus description thereof is omitted. Furthermore, since the processing of step S19a, step S23a, and steps S31 to S35 illustrated in FIG. 29 is the same as that in FIG. 21, the description thereof will be omitted.

Then, in step S35, when the blood vessel width candidate is not likely (step S35: No), the acquisition function 384b acquires a blood vessel width candidate based on the velocity information as the fourth blood vessel width candidate (step S41). Specifically, the acquisition function 384b acquires the blood vessel width candidate based on the velocity information as the fourth blood vessel width candidate by executing the blood vessel width candidate acquiring processing based on the velocity information described above.

Next, as illustrated in FIG. 29, the adjustment function 385 in the processing circuitry 38 of the apparatus main body 30 compares the first blood vessel width candidate with the fourth blood vessel width candidate (step S43). Specifically, the adjustment function 385 compares, based on the plurality of blood vessel width candidates, a plurality of blood vessel width candidates. More specifically, the adjustment function 385 compares the blood vessel width candidate based on the examination information acquired as the first blood vessel width candidate in step S17 with the blood vessel width candidate based on the velocity information acquired as the fourth blood vessel width candidate in step S41. More specifically, for example, the adjustment function 385 compares whether the blood vessel width candidate based on the velocity information is within the range of the blood vessel width candidate based on the examination information.

Next, as illustrated in FIG. 29, the adjustment function 385 determines whether or not the blood vessel width candidate is likely (step S45). Specifically, the adjustment function 385 determines whether or not the blood vessel width candidate is likely based on a comparison result that is a result of comparing the plurality of blood vessel width candidates. More specifically, the adjustment function 385 determines whether or not the blood vessel width candidate based on the velocity information is likely based on a result of comparing the blood vessel width candidate based on the examination information acquired as the first blood vessel width candidate with the blood vessel width candidate based on the velocity information acquired as the fourth blood vessel width candidate in step S43. More specifically, for example, when at least one of the first blood vessel width candidate and the fourth blood vessel width candidate has an error value, the adjustment function 385 determines that the blood vessel width candidate is not likely. Furthermore, for example, when the blood vessel width candidate based on the velocity information is not within the range of the blood vessel width candidate based on the examination information, the adjustment function 385 determines that it is not likely. On the other hand, for example, when the blood vessel width candidate based on the velocity information is within the range of the blood vessel width candidate based on the examination information, the adjustment function 385 determines that the blood vessel width candidate is likely.

Then, in step S45, when the blood vessel width candidate is not likely (step S45: No), the acquisition function 384b acquires a blood vessel width candidate based on the dispersion information as the fifth blood vessel width candidate (step S47). Specifically, the acquisition function 384b acquires the blood vessel width candidate based on the dispersion information as the fifth blood vessel width candidate by executing the blood vessel width candidate acquiring processing based on the dispersion information described above.

Next, as illustrated in FIG. 29, the adjustment function 385 compares the first blood vessel width candidate with the fifth blood vessel width candidate (step S49). Specifically, the adjustment function 385 compares a plurality of blood vessel width candidates based on the plurality of blood vessel width candidates. More specifically, the adjustment function 385 compares the blood vessel width candidate based on the examination information acquired as the first blood vessel width candidate in step S17 with the blood vessel width candidate based on the dispersion information acquired as the fifth blood vessel width candidate in step S47. For example, the adjustment function 385 compares whether the blood vessel width candidate based on the dispersion information is within the range of the blood vessel width candidate based on the examination information.

Next, as illustrated in FIG. 29, the adjustment function 385 determines whether or not the blood vessel width candidate is likely (step S51). Specifically, the adjustment function 385 determines whether or not the blood vessel width candidate is likely based on a comparison result that is a result of comparing the plurality of blood vessel width candidates. More specifically, the adjustment function 385 determines whether or not the blood vessel width candidate based on the dispersion information is likely based on a result of comparing the blood vessel width candidate based on the examination information acquired as the first blood vessel width candidate with the blood vessel width candidate based on the dispersion information acquired as the fifth blood vessel width candidate in step S49. More specifically, for example, when at least one of the first blood vessel width candidate and the fifth blood vessel width candidate has an error value, the adjustment function 385 determines that the blood vessel width candidate is not likely. Furthermore, for example, when the blood vessel width candidate based on the dispersion information is not within the range of the blood vessel width candidate based on the examination information, the adjustment function 385 determines that it is not likely. On the other hand, for example, when the blood vessel width candidate based on the dispersion information is within the range of the blood vessel width candidate based on the examination information, the adjustment function 385 determines that the blood vessel width candidate is likely.

Then, in steps S23a, S35, S45, and S51, when the blood vessel width candidate is likely (steps S23a, S35, S45, and S51: Yes), the width of the sample volume SV is adjusted (step S25). Note that the specific processing in step S25 is the same as that in FIG. 13, and thus the description thereof will be omitted.

On the other hand, in step S51, when the blood vessel width candidate is not likely (step S51: No), it is determined whether or not max(X, Y) and min(X, Y) in the blood vessel width candidate based on the B mode information are errors (step S53). Specifically, the acquisition function 384b determines whether or not an error value is stored in max(X, Y) and min(X, Y) in the blood vessel width candidate based on the B mode information acquired in step S19a.

Then, when max(X, Y) and min(X, Y) in the blood vessel width candidate based on the B mode information are not errors in step S53 (step S53: No), the acquisition function 384b adjusts the width of the sample volume SV based on one of max(X, Y) and min(X, Y) in the blood vessel width candidate based on the B mode information and the blood vessel width candidate based on the examination information (step S55). Specifically, the acquisition function 384b adjusts the width of the sample volume SV based on the blood vessel width candidate based on the examination information acquired in step S17 and one of max(X, Y) and min(X, Y) stored by executing the blood vessel width candidate acquiring processing based on the B mode information in step S19.

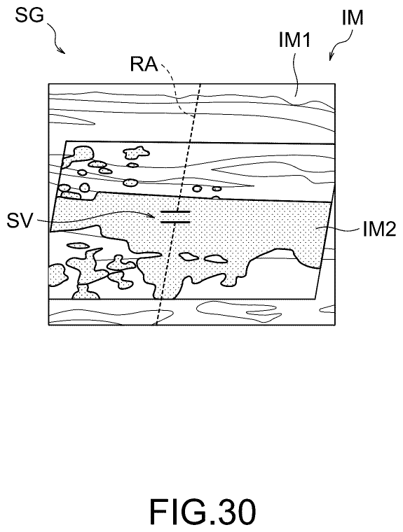

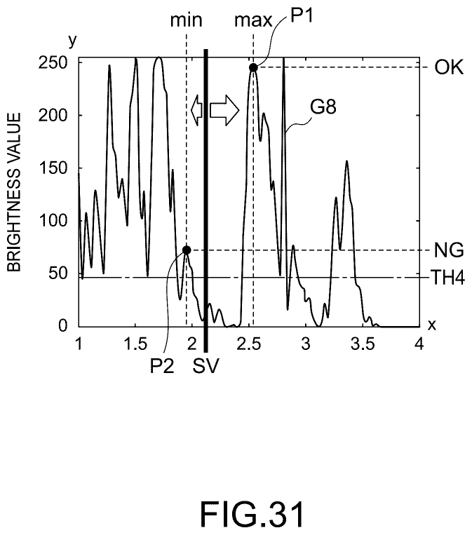

A method of adjusting the width of the sample volume SV in step S55 will be described in detail with reference to FIGS. 30 and 31. FIG. 30 is a diagram illustrating an example of the ultrasonic image IM in which the sample gate SG is set. FIG. 31 is a diagram illustrating an example of distribution of brightness values on the raster RA acquired in the ultrasonic image IM illustrated in FIG. 30. As illustrated in FIG. 30, the sample gate SG is set in the B mode image IM1 which is the ultrasonic image IM. The sample volume SV included in the sample gate SG is set on the blood vessel BV depicted on the B mode image IM1. In addition, a color Doppler image IM2 is superimposed on the B mode image IM1.

As illustrated in FIG. 31, the acquisition function 384b acquires a graph G8 in which the x-axis is Depth and the y-axis is a brightness value as the distribution of the brightness value on the raster RA in the sample gate SG of the ultrasonic image IM illustrated in FIG. 30. In addition, the acquisition function 384b stores the coordinates at the point P1 stored by executing the blood vessel width candidate acquiring processing based on the B mode information as max(X, Y), and stores the coordinates at the point P2 as min(X, Y). Then, the acquisition function 384b assumes that one of stored max(X, Y) and min(X, Y) has a larger difference between the brightness value and the threshold more correctly captures the blood vessel wall. For example, in the example illustrated in FIG. 31, the point P1 has a large difference between the brightness value and the threshold TH4, and thus it is assumed that max(X, Y) stored as coordinates at the point P1 captures the blood vessel wall more correctly.

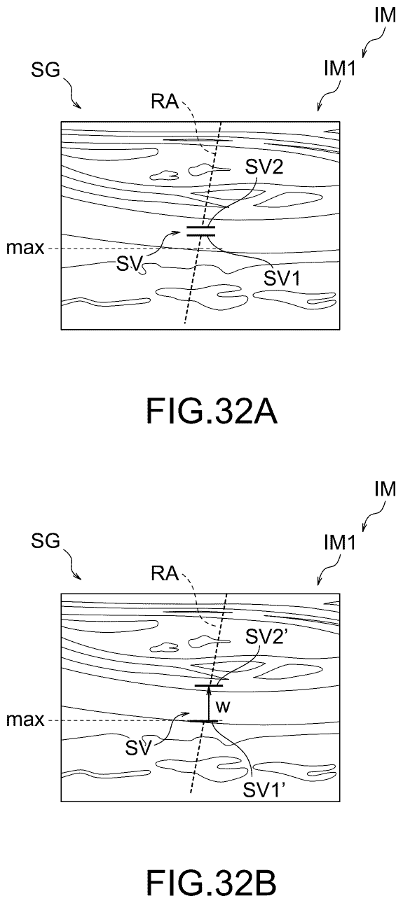

FIGS. 32A and 32B are diagrams illustrating an example of the ultrasonic image IM in which the sample gate SG including the sample volume SV before and after adjustment of the width of the sample volume SV is set. As illustrated in FIG. 32A, before adjustment by the adjustment function 385, the sample volume SV does not have an appropriate width with respect to the blood vessel width. On the other hand, as illustrated in FIG. 32B, after the adjustment by the adjustment function 385, the sample volume SV′ has an appropriate width with respect to the blood vessel width. Specifically, as illustrated in FIG. 32B, the adjustment function 385 adjusts the width of the sample volume SV by aligning the lower end SV1 of the sample gate with the position of max(X, Y) and opening the upper end SV2 of the sample volume SV from the lower end SV1 of the sample volume SV by the value W of the blood vessel width candidate based on the search information.

Then, in step S53, when the blood vessel width candidate is not likely (step S23: No), and when max(X, Y) and min(X, Y) in the blood vessel width candidate based on the B mode information are errors (step S53: Yes), the sample volume width adjustment processing is ended after the processing of step S25 or after the processing of step S55.

As described above, in the ultrasonic diagnostic apparatus 1, even when none of the blood vessel width candidates based on other information is likely based on the blood vessel width candidate based on the examination information, the sample volume SV included in the sample gate SG is adjusted based on one of max(X, Y) and min(X, Y) in the blood vessel width candidate based on the B mode information and the blood vessel width candidate based on the examination information. Therefore, it is not necessary to manually adjust the sample volume SV for each blood vessel, and the sample volume SV can be appropriately adjusted according to the blood vessel width, so that the examination efficiency and the examination accuracy in the PWD mode can be improved.

Note that, in steps S53 and S55 of the sample volume width adjustment processing executed in the ultrasonic diagnostic apparatus 1 according to the third embodiment described above, the acquisition function 384b determines whether or not max(X, Y) and min(X, Y) in the blood vessel width candidate based on the B mode information are errors, and adjusts the width of the sample volume SV based on one of max(X, Y) and min(X, Y) in the blood vessel width candidate based on the B mode information and the blood vessel width candidate based on the examination information when the max and min are not errors. However, the blood vessel width candidate for which the acquisition function 384b determines whether or not max(X, Y) and min(X, Y) are errors is not limited to the B mode information. That is, the blood vessel width candidates for which the acquisition function 384b determines whether or not max(X, Y) and min(X, Y) are errors are arbitrary, it is determined whether or not max and min in any one of the blood vessel width candidate based on the power information, the blood vessel width candidate based on the velocity information, and the blood vessel width candidate based on the dispersion information are errors, and when max(X, Y) and min(X, Y) are not errors, the width of the sample volume SV may be adjusted based on any one of max(X, Y) and min(X, Y) in any one of the blood vessel width candidate based on the power information, the blood vessel width candidate based on the velocity information, and the blood vessel width candidate based on the dispersion information and the blood vessel width candidate based on the examination information.

### Fourth Embodiment

In the first to third embodiments described above, the case where the blood vessel width candidate based on the examination information can be used has been described, but it is not limited thereto. In a fourth embodiment, an ultrasonic diagnostic apparatus will be described that determines whether or not a blood vessel width based on examination information can be used, and adjusts a sample volume SV from a blood vessel width candidate based on information other than the examination information when the blood vessel width candidate based on the examination information cannot be used. Hereinafter, a case where a modification is applied to the first embodiment described above will be described as a fourth embodiment, but the modification can also be applied to the second and third embodiments. Note that the configuration of the ultrasonic diagnostic apparatus 1 is the same as that in FIG. 1, and thus description thereof will be omitted.

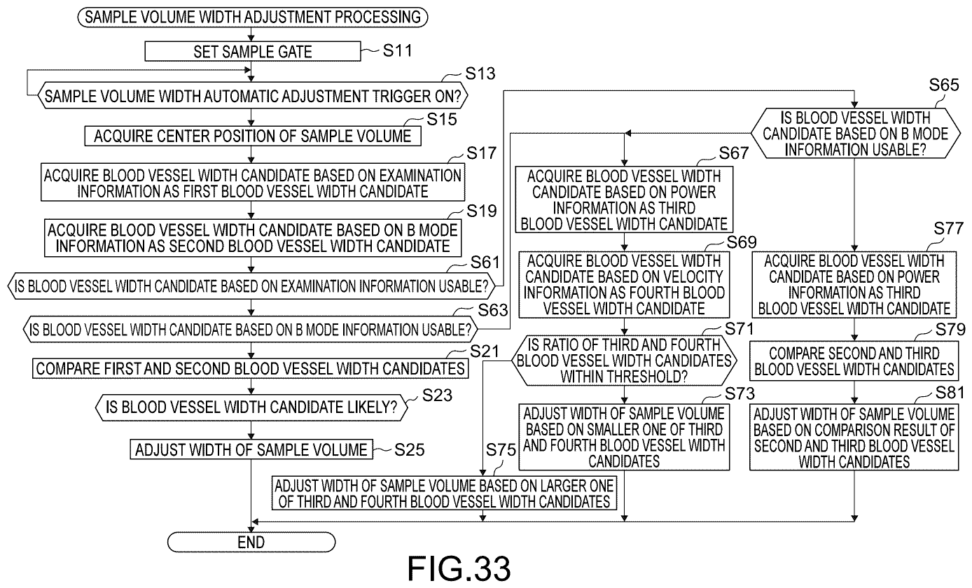

FIG. 33 is a flowchart for describing the sample volume width adjustment processing executed in the ultrasonic diagnostic apparatus 1 according to the third embodiment, and is a diagram corresponding to FIG. 13. In the sample volume width adjustment processing, the sample gate SG is set, the center position of the sample volume SV is acquired, the blood vessel width candidate is acquired based on the examination information, the blood vessel width candidate is acquired based on the B mode information, whether or not the blood vessel width candidate based on the examination information and the blood vessel width candidate based on the B mode information are usable is determined, the blood vessel width candidate is acquired based on the power information, the blood vessel width candidate is acquired based on the velocity information, and the width of the sample volume SV is adjusted. For example, the sample volume width adjustment processing is executed when the PWD mode is activated.

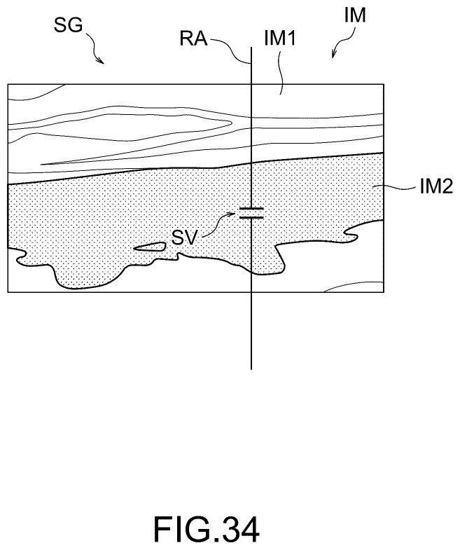

As illustrated in FIG. 33, first, the sample gate setting function 383 in the processing circuitry 38 of the apparatus main body 30 sets a sample gate (step S11). Specific processing is the same as step S11 in FIG. 13, and thus description thereof will be omitted. FIG. 34 is a diagram illustrating an example of the ultrasonic image IM in which the sample gate SG is set, and is a diagram corresponding to FIG. 14. As illustrated in FIG. 34, a sample gate SG is set in the color Doppler image IM2 superimposed on the B mode image IM1 which is the ultrasonic image IM. Note that the processing in steps S13 to S19 after step S11 illustrated in FIG. 33 is the same as that in FIG. 13, and thus description thereof is omitted.

Next, as illustrated in FIG. 33, the acquisition function 384 determines whether or not the blood vessel width candidate based on the examination information can be used (step S61). Specifically, the acquisition function 384 determines whether or not the blood vessel width candidate based on the examination information is usable by determining whether or not the blood vessel width candidate based on the examination information acquired in step S17 includes an error value.

Then, in step S61, when the blood vessel width candidate based on the examination information is usable (step S61: Yes), the acquisition function 384 determines whether or not the blood vessel width candidate based on the B mode information is usable (step S63). Specifically, the acquisition function 384 determines whether or not the B mode information is usable by determining whether or not the blood vessel width candidate based on the B mode information includes an error value.

On the other hand, when the blood vessel width candidate based on the examination information is unusable in step S61 (step S61: No), the acquisition function 384 determines whether or not the blood vessel width candidate based on the B mode information is usable (step S65). Specifically, the acquisition function 384 determines whether or not the B mode information is usable by determining whether or not the blood vessel width candidate based on the B mode information includes an error value.

Then, in steps S63 and S65, when the blood vessel width candidate based on the B mode information is not available (steps S63 and S65: No), the acquisition function 384 acquires the blood vessel width candidate based on the power information as the third blood vessel width candidate (step S67). Specifically, the acquisition function 384 acquires the blood vessel width candidate based on the power information as the third blood vessel width candidate by executing the blood vessel width candidate acquiring processing based on the power information.

Next, the acquisition function 384 acquires a blood vessel width candidate based on the velocity information as a fourth blood vessel width candidate (step S69). Specifically, the acquisition function 384 acquires the blood vessel width candidate based on the velocity information as the fourth blood vessel width candidate by executing the blood vessel width candidate acquiring processing based on the velocity information.

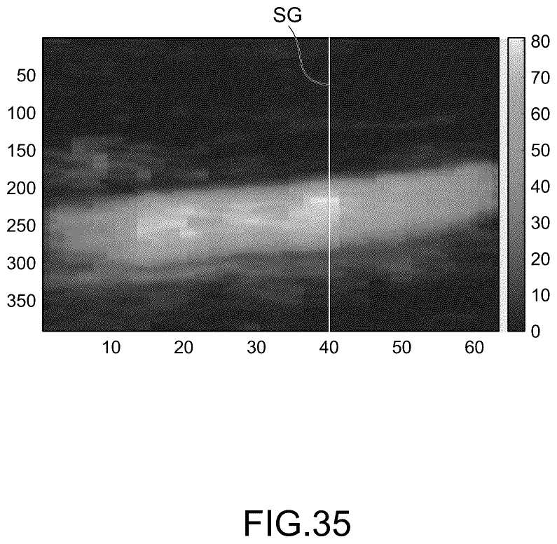

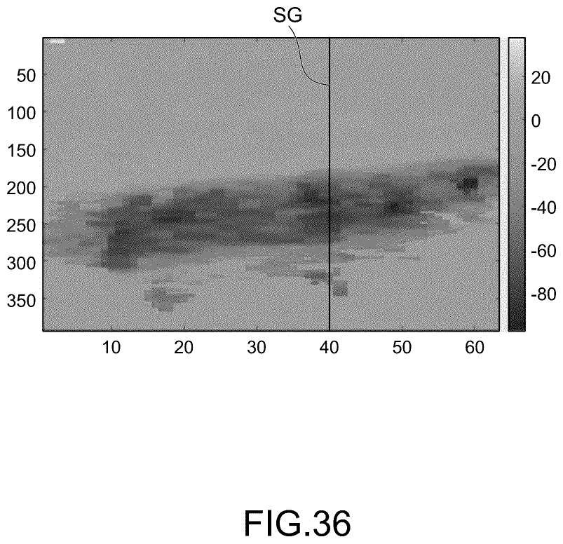

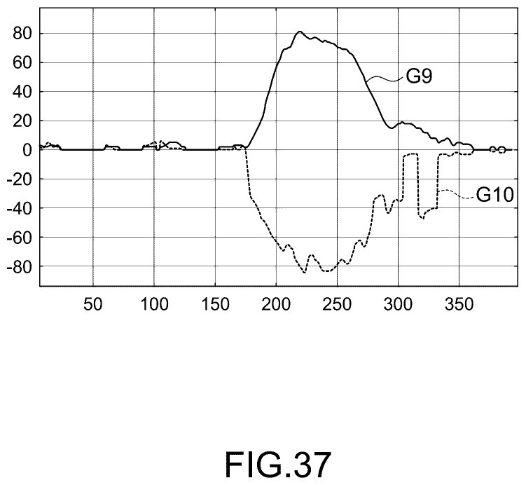

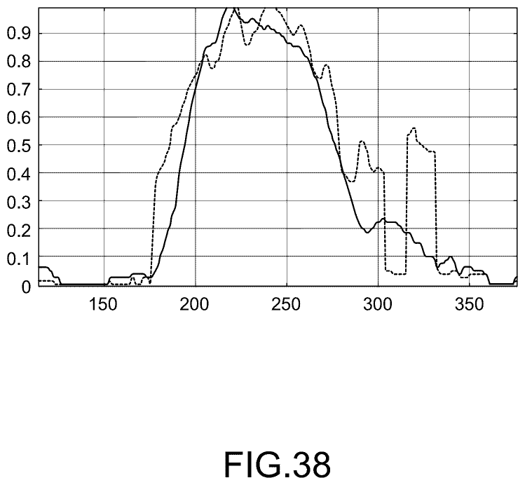

The power information and the velocity information acquired in the present embodiment will be described with reference to FIGS. 35, 36, 37, and 38. FIG. 35 is a diagram illustrating an example of a distribution of power values in the ultrasonic image IM illustrated in FIG. 34. FIG. 36 is a diagram illustrating an example of a distribution of velocity values in the ultrasonic image IM illustrated in FIG. 34. FIG. 37 is a diagram illustrating an example of the distribution of power values on the raster RA illustrated in FIG. 35 and the distribution of velocity values on the raster RA illustrated in FIG. 36. FIG. 38 is a diagram obtained by normalizing each of the distribution of power values and the distribution of velocity values illustrated in FIG. 37.

The distribution of power values on the raster in the sample gate SG set to the distribution of power values illustrated in FIG. 35 is indicated by a graph G9 illustrated in FIG. 37. Further, the distribution of the velocity values on the raster in the sample gate SG set to the distribution of the velocity values illustrated in FIG. 36 is indicated by a graph G10 illustrated in FIG. 37. A graph obtained by normalizing each of the graph G9 and the graph G10 is illustrated in FIG. 38. Then, by executing the blood vessel width candidate acquiring processing based on the power information and the blood vessel width candidate acquiring processing based on the velocity information, the acquisition function 384 acquires the blood vessel width candidate based on the power information in step S67 and acquires the blood vessel width candidate based on the velocity information in step S69. Specifically, the acquisition function 384 acquires the blood vessel width candidate based on the power information and the blood vessel width candidate based on the velocity information by obtaining a point equal to or less than a certain threshold from a peak value closest to the center position of the sample volume SV based on the graph illustrated in FIG. 38.

Next, as illustrated in FIG. 33, the adjustment function 385 determines whether or not the ratio between the third and fourth blood vessel width candidates is within a threshold (step S71). Specifically, based on the blood vessel width candidate based on the power information acquired as the third blood vessel width candidate in step S67 and the blood vessel width candidate based on the velocity information acquired as the fourth blood vessel width candidate in step S69, the adjustment function 385 determines whether or not the ratio between the blood vessel width candidate based on the power information and the blood vessel width candidate based on the velocity information is within the threshold. More specifically, the adjustment function 385 determines whether or not a value obtained by dividing a larger one of the blood vessel width candidate based on the power information and the blood vessel width candidate based on the velocity information by a smaller one of the blood vessel width candidate based on the power information and the blood vessel width candidate based on the velocity information is within a threshold.

Then, in step S71, when the ratio between the third and fourth blood vessel width candidates is within the threshold (step S71: Yes), the adjustment function 385 adjusts the width of the sample volume SV based on the smaller one of the third and fourth blood vessel width candidates (step S73). Specifically, the adjustment function 385 adjusts the width of the sample volume SV based on max(X, Y) and min(X, Y) in the smaller one of the third and fourth blood vessel width candidates.

On the other hand, in step S71, when the ratio between the third and fourth blood vessel width candidates is not within the threshold (step S71: No), the adjustment function 385 adjusts the width of the sample volume SV based on the larger one of the third and fourth blood vessel width candidates (step S75). Specifically, the adjustment function 385 adjusts the width of the sample volume SV based on max(X, Y) and min(X, Y) in the larger one of the third and fourth blood vessel width candidates.

On the other hand, in step S65, when the blood vessel width candidate based on the B mode information is usable (step S65: Yes), the acquisition function 384 acquires the blood vessel width candidate based on the power information as the third blood vessel width candidate (step S77). Specifically, by executing the blood vessel width candidate acquiring processing based on the power information, the acquisition function 384 acquires the blood vessel width candidate based on the power information as the third blood vessel width candidate.

Next, as illustrated in FIG. 33, the adjustment function 385 compares the second and third blood vessel width candidates (step S77). Specifically, the adjustment function 385 compares the blood vessel width candidate based on the B mode information acquired in step S19 with the blood vessel width candidate based on the power information acquired in step S77.

More specifically, the adjustment function 385 compares max(X, Y) in the blood vessel width candidate based on the B mode information with max(X, Y) in the blood vessel width candidate based on the power information, and when max(X, Y) in the blood vessel width candidate based on the B mode information is farther from the coordinates (X_{c}, Y_{c}) of the center position of the sample volume SV than max(X, Y) in the blood vessel width candidate based on the power information and the blood vessel width candidate based on the B mode information is larger than the blood vessel width candidate based on the power information, the adjustment function 385 uses max(X, Y) in the blood vessel width candidate based on the power information. On the other hand, other than the case where max(X, Y) in the blood vessel width candidate based on the B mode information is farther from the coordinates (X_{c}, Y_{c}) of the center position of the sample volume SV than max(X, Y) in the blood vessel width candidate based on the power information, and the blood vessel width candidate based on the B mode information is larger than the blood vessel width candidate based on the power information, for example, in a case where max(X, Y) in the blood vessel width candidate based on the B mode information is closer to the coordinates (X_{c}, Y_{c}) of the center position of the sample volume SV than max(X, Y) in the blood vessel width candidate based on the power information, the adjustment function 385 uses max(X, Y) in the blood vessel width candidate based on the B mode information. The adjustment function 385 also derives min(X, Y) similarly to max(X, Y).

Next, the adjustment function 385 adjusts the width of the sample volume SV based on the comparison result between the second and third blood vessel width candidates (step S81). Specifically, the adjustment function 385 adjusts the width of the sample volume SV based on max(X, Y) and min(X, Y) derived as a comparison result in step S79.

As described above, in the ultrasonic diagnostic apparatus 1 according to the fourth embodiment, even when the blood vessel width candidate based on the examination information cannot be used, a plurality of blood vessel width candidates based on information other than the examination information is acquired, and the width of the sample volume SV is adjusted based on the plurality of blood vessel width candidates. Therefore, it is not necessary to manually adjust the sample volume SV for each blood vessel, and the sample volume SV can be appropriately adjusted according to the blood vessel width, so that the examination efficiency and the examination accuracy in the PWD mode can be improved.

### [Modification 1]

In the ultrasonic diagnostic apparatus 1 according to the first to fourth embodiments described above, the adjustment function 385 may adjust the sample volume SV according to the ratio of the width of the sample volume SV to the blood vessel width set in advance. Specifically, the adjustment function 385 may adjust the width of the sample volume SV using the following Expressions (4) and (5) in consideration of the ratio of the width of the sample volume SV to the blood vessel width set in advance by the user (ratio of user setting) with respect to Expressions (1) and (2) described above.

$$\begin{matrix}[ Math . 4 ] & \\ Extension rate of upper end = \frac{( {Y}_{c} - {Y}_{min} ) \times ( Ratio of user setting )}{W / 2} & ( 4 )\end{matrix}$$

$$\begin{matrix}[ Math . 5 ] & \\ Extension rate of lower end = \frac{( {Y}_{max} - {Y}_{c} ) \times ( Ratio of user setting )}{W / 2} & ( 5 )\end{matrix}$$

In this manner, by adjusting the width of the sample volume SV using the following Expressions (4) and (5) in consideration of the ratio of the width of the sample volume SV to the blood vessel width set by the user, the possibility that the upper end SV2 and the lower end SV1 of the sample volume SV can be arranged within the width of the blood vessel BV in the ultrasonic image IM is increased, and thus it is possible to reduce the possibility that a folding phenomenon occurs due to the edge of the blood vessel BV or the like being included within the width of the sample volume SV.

### [Other Modifications]

In the ultrasonic diagnostic apparatus 1 according to the first embodiment described above, the acquisition function 384 acquires the blood vessel width candidate based on the B mode information as the second blood vessel width candidate, but the blood vessel width candidate acquired by the acquisition function 384 as the second blood vessel width candidate is not limited thereto. That is, the blood vessel width candidate acquired as the second blood vessel width candidate by the acquisition function 384 is arbitrary, and for example, the acquisition function 384 may acquire, as the second blood vessel width candidate, a blood vessel width candidate based on power information, a blood vessel width candidate based on velocity information, or a blood vessel width candidate based on dispersion information.

In the ultrasonic diagnostic apparatus 1 according to the first embodiment described above, the acquisition function 384 acquires the blood vessel width candidate based on the examination information as the first blood vessel width candidate, and acquires the blood vessel width candidate based on the B mode information as the second blood vessel width candidate, but the combination of the first blood vessel width candidate and the second blood vessel width candidate acquired by the acquisition function 384 is not limited thereto. That is, the combination of the first blood vessel width candidate and the second blood vessel width candidate acquired by the acquisition function 384 is arbitrary, and the acquisition function 384 may acquire, as the first blood vessel width candidate, any one of the blood vessel width candidate based on the examination information, the blood vessel width candidate based on the B mode information, the blood vessel width candidate based on the power information, the blood vessel width candidate based on the velocity information, and the blood vessel width candidate based on the dispersion information, and acquire, as the second blood vessel width candidate, a blood vessel width candidate different from the first blood vessel width candidate. Note that, when the acquisition function 384 does not acquire the blood vessel width candidate based on the examination information as the first blood vessel width candidate and the second blood vessel width candidate, the processing of step S21 and the processing of step S23 in the sample volume width adjustment processing may be omitted. In this case, in step S25, the adjustment function 385 may adjust the width of the sample volume SV based on the first blood vessel width candidate and the second blood vessel width candidate acquired in steps S17 and S19.

Further, in the ultrasonic diagnostic apparatus 1 according to the first embodiment described above, the acquisition function 384 acquires two blood vessel width candidates, but it is not limited thereto. That is, the acquisition function 384 may acquire three or more blood vessel width candidates. In this case, the adjustment function 385 may adjust the region included in the sample gate SG based on three or more blood vessel width candidates.

Further, in the ultrasonic diagnostic apparatus 1 according to the second embodiment described above, the acquisition function 384a acquires the blood vessel width candidate based on the power information as the third blood vessel width candidate, but the third blood vessel width candidate acquired by the acquisition function 384a is not limited thereto. That is, the third blood vessel width candidate acquired by the acquisition function 384a is arbitrary, and for example, the acquisition function 384a may acquire the blood vessel width candidate based on the velocity information or the blood vessel width candidate based on the dispersion information as the third blood vessel width candidate.

In addition, in the ultrasonic diagnostic apparatus 1 according to the third embodiment described above, in step S55, when one of max(X, Y) and min(X, Y) in any of the blood vessel width candidate based on the B mode information, the blood vessel width candidate based on the power information, the blood vessel width candidate based on the velocity information, and the blood vessel width candidate based on the dispersion information includes an error value, the width of the sample volume SV may be adjusted based on the other of max(X, Y) and min(X, Y) in any of the blood vessel width candidate based on the B mode information, the blood vessel width candidate based on the power information, the blood vessel width candidate based on the velocity information, and the blood vessel width candidate based on the dispersion information and the blood vessel width candidate based on the examination information.

Furthermore, in the ultrasonic diagnostic apparatus 1 according to the third embodiment described above, in step S53, when max(X, Y) and min(X, Y) in all of the blood vessel width candidates based on the B mode information, the blood vessel width candidates based on the power information, the blood vessel width candidates based on the velocity information, and the blood vessel width candidates based on the dispersion information include error values, the adjustment function 385 may adjust the width of the sample volume SV only based on the blood vessel width candidates based on the examination information. In this case, the adjustment function 385 may adjust the width of the sample volume SV by setting positions moved from the center position of the sample volume by the blood vessel width candidate/2 based on the examination information as max(X, Y) and min(X, Y) in each of the positive direction and the negative direction of the y coordinate.

Note that, in the ultrasonic diagnostic apparatus 1 according to the fourth embodiment described above, the acquisition function 384b acquires the blood vessel width candidate based on the power information as the third blood vessel width candidate, and acquires the blood vessel width candidate based on the velocity information as the fourth blood vessel width candidate, but the third and fourth blood vessel width candidates acquired by the acquisition function 384b are not limited thereto. That is, the third and fourth blood vessel width candidates acquired by the acquisition function 384b are arbitrary, and for example, the acquisition function 384b may acquire the blood vessel width candidate based on the velocity information or the blood vessel width candidate based on the dispersion information as the third blood vessel width candidate, or may acquire the blood vessel width candidate based on the power information or the blood vessel width candidate based on the dispersion information as the fourth blood vessel width candidate.

In the ultrasonic diagnostic apparatus 1 according to the first to fourth embodiments described above, the sample gate setting function 383 sets the sample gate SG in the ultrasonic image IM during execution of the PWD mode scan, and then the acquisition function 384, 384a, or 384b acquires a plurality of blood vessel width candidates when receiving a press of the sample volume width adjustment button which is the first input operation related to the adjustment of the sample volume, but it is not limited thereto. That is, the timing of acquiring the plurality of blood vessel width candidates is arbitrary. For example, when the sample gate setting function 383 receives a second input operation related to the movement of the sample volume SV from the user during the execution of the PWD mode scan after receiving the press of the sample volume width adjustment button which is the first input operation related to the adjustment of the sample volume, the acquisition function 384, 384a, or 384b may acquire a plurality of blood vessel width candidates. In addition, for example, when the sample gate setting function 383 receives a third input operation related to the start of execution of the PWD mode scan from the user after receiving a press of the sample volume width adjustment button which is the first input operation related to the adjustment of the sample volume SV, the acquisition function 384, 384a, or 384b may acquire a plurality of blood vessel width candidates.

Further, in the ultrasonic diagnostic apparatus 1 according to the first to fourth embodiments described above, the width of the sample volume SV is adjusted using the above-described Expressions (1) and (2), but the method of adjusting the width of the sample volume SV is not limited thereto. For example, the adjustment function 385 may adjust the width of the sample volume SV so as to arrange the lower end SV1 of the sample volume SV and the upper end SV2 of the sample volume SV at the positions of max(X, Y) and min(X, Y) based on max(X, Y) and min(X, Y) in the blood vessel width candidate based on the B mode information determined to be within the range of the blood vessel width candidate based on the examination information in step S23. In addition, in the ultrasonic diagnostic apparatus 1 according to Modification 1, the width of the sample volume SV may be adjusted such that the lower end SV1 of the sample volume SV and the upper end SV2 of the sample volume SV are arranged at the positions of max(X, Y) and min(X, Y) according to the ratio of the width of the sample volume SV to the blood vessel width preset by the user.

In addition, in the ultrasonic diagnostic apparatus according to the first to fourth embodiments and Modification 1 described above, the acquisition function 384, 384a, or 384b estimates the examination site from the information regarding the examination site included in the setting contents of the examination mode included in the examination information, but it is not limited thereto. The acquisition function 384, 384a, or 384b may estimate the examination site from information such as Depth and the depth and size of the region of interest included in the examination information, or may estimate the examination site by analyzing the B mode image IM1 displayed on the display 70 using AI. In this manner, by enabling estimation of the examination site using AI and information such as Depth and the depth and size of the region of interest included in the examination information, it is possible to estimate the examination site even when an examination different from the examination information such as examination of the lower limb blood vessels is performed in a case where the setting content of the examination mode is abdominal examination.

In the ultrasonic diagnostic apparatus 1 according to the first to fourth embodiments and Modification 1 described above, when the blood vessel width candidate is not within the range of the blood vessel width candidate based on the examination information, it is determined that the blood vessel width candidate is not likely and the width of the sample volume SV is not adjusted, but it is not limited thereto. For example, when the blood vessel width candidate based on the B mode information is not within the range of the blood vessel width candidate based on the examination information, the ultrasonic diagnostic apparatus 1 may adjust the sample volume SV based on any narrower blood vessel width candidate. When the narrower one of the blood vessel width candidates is the blood vessel width candidate based on the examination information, the adjustment function 385 may adjust the width of the sample volume SV by setting, as max(X, Y) and min(X, Y), coordinates of a position where the y coordinate is moved by the blood vessel width candidate/2 based on the examination information in each of the positive direction and the negative direction from the coordinates of the center position of the sample volume SV. Note that, although the case where the blood vessel width candidate based on the B mode information is not within the range of the blood vessel width candidate based on the examination information has been described as an example, the same applies to the case of the blood vessel width candidate based on the power information, the blood vessel width candidate based on the velocity information, and the blood vessel width candidate based on the dispersion information instead of the blood vessel width candidate based on the B mode information.

In addition, in the ultrasonic diagnostic apparatus 1 according to the first to fourth embodiments and Modification 1 described above, the acquisition function 384, 384a, or 384b may acquire priority order information and acquire a plurality of blood vessel width candidates based on at least one of the reception information and the examination information based on the priority order information. The priority order information is information related to the priority order of the reception information of ultrasonic waves and the examination information. The priority order information includes, for example, information regarding an order of acquisition of a blood vessel width candidate based on a velocity value of blood flow, a blood vessel width candidate based on a dispersion value of blood flow, a blood vessel width candidate based on a power value of blood flow, a blood vessel width candidate based on a brightness value in the B mode information, and the blood vessel width candidate based on the examination information.

Note that the word “processor” used in above descriptions means circuits such as, for example, a Central Processing Unit (CPU), a Graphics Processing Unit (GPU), an Application Specific Integrated Circuit (ASIC), a programmable logic device (for example, a Simple Programmable Logic Apparatus (SPLD), a Complex Programmable Logic Apparatus (CPLD), and a Field Programmable Gate Array (FPGA)). The processor executes functions by reading and executing programs stored in the memory 37. Note that programs may be configured to be directly integrated in the processor instead of being storing in the memory 37. In this case, the processor realizes functions by reading and executing programs stored in the circuitry. Note that the processor is not limited to the case arranged as a single processor circuit, but may be configured as a single processor by combining a plurality of independent circuits to realize functions. Furthermore, a plurality of component elements in FIG. 1 may be integrated into one processor to realize the functions.

According to at least one embodiment described above, the examination efficiency and the examination accuracy in the PWD mode can be improved.

While certain embodiments have been described, these embodiments have been presented by way of example only and are not intended to limit the scope of the inventions. The embodiments may be in a variety of other forms. Furthermore, various omissions, substitutions and changes may be made without departing from the spirit of the inventions. The embodiments and their modifications are included in the scope and the subject matter of the invention, and at the same time included in the scope of the claimed inventions and their equivalents.

1. An ultrasonic diagnostic apparatus comprising:
processing circuitry configured to
set a sample gate including a region where blood flow information of a subject is acquired on an ultrasonic image of the subject;
acquire, based on at least one of reception information of ultrasonic waves of the subject and examination information, a plurality of blood vessel width candidates for a blood vessel at a position where the sample gate is set; and
adjust the region included in the sample gate based on the plurality of blood vessel width candidates.

2. The ultrasonic diagnostic apparatus of claim 1, wherein the reception information includes power information based on a reception signal of the ultrasonic wave, dispersion information based on the reception signal, velocity information based on the reception signal, and B mode information based on the reception signal, the power information being information regarding power of blood flow, the dispersion information being information regarding dispersion of blood flow, the velocity information being information regarding velocity of blood flow, and the B mode information being information regarding B mode and
the processing circuitry is further configured to acquire, based on at least any two pieces of information of the power information, the dispersion information, the velocity information, the B mode information, and the examination information, blood vessel width candidates based on each of at the least any two pieces of information as the plurality of blood vessel width candidates.

3. The ultrasonic diagnostic apparatus of claim 1, wherein the processing circuitry is further configured to
set the sample gate in the ultrasonic image obtained by imaging the blood vessel from one imaging direction; and
acquire, based on at least one of the reception information of ultrasonic waves of the subject and the examination information, the plurality of blood vessel width candidates for the blood vessel in the ultrasonic image obtained by imaging the blood vessel from one imaging direction.

4. The ultrasonic diagnostic apparatus of claim 1, wherein the processing circuitry is further configured to
acquire priority order information related to the reception information of ultrasonic waves of the subject and the examination information; and
acquire, based on the priority order information, the plurality of blood vessel width candidates based on at least one of the reception information and the examination information.

5. The ultrasonic diagnostic apparatus of claim 1, wherein the processing circuitry is further configured to
compare the plurality of blood vessel width candidates based on the plurality of blood vessel width candidates;
determine whether or not the blood vessel width candidate is likely based on a comparison result that is a result of comparing the plurality of blood vessel width candidates; and
adjust the region included in the sample gate when it is determined that the blood vessel width candidate is likely.

6. The ultrasonic diagnostic apparatus of claim 2, wherein the processing circuitry is further configured to
acquire, as the plurality of blood vessel width candidates, a blood vessel width candidate based on the B mode information and a blood vessel width candidate based on the examination information;
compare, based on the blood vessel width candidate based on the B mode information and the blood vessel width candidate based on the examination information, the blood vessel width candidate based on the B mode information with the blood vessel width candidate based on the examination information;
determine, based on a comparison result between the blood vessel width candidate based on the B mode information and the blood vessel width candidate based on the examination information, whether or not the blood vessel width candidate based on the B mode information is likely; and
adjust, based on the blood vessel width candidate based on the B mode information, the region when it is determined that the blood vessel width candidate based on the B mode information is likely.

7. The ultrasonic diagnostic apparatus of claim 6, wherein the processing circuitry is further configured to
compare a blood vessel width candidate based on any one of the power information, the dispersion information, and the velocity information with the blood vessel width candidate based on the examination information when it is determined that the blood vessel width candidate based on the B mode information is not likely; and
determine, based on a comparison result between the blood vessel width candidate based on any one of the power information, the dispersion information, and the velocity information and the blood vessel width candidate based on the examination information, whether or not the blood vessel width candidate based on any one of the power information, the dispersion information, and the velocity information is likely.

8. The ultrasonic diagnostic apparatus of claim 1, wherein the reception information is raw data before scan conversion processing.

9. The ultrasonic diagnostic apparatus of claim 1, wherein the processing circuitry is further configured to receive a first input operation related to adjustment of the region from a user.

10. The ultrasonic diagnostic apparatus of claim 9, wherein the processing circuitry is further configured to acquire the plurality of blood vessel width candidates when the first input operation is received after the sample gate is set to the ultrasonic image during execution of a PWD method scan that transmits a pulse wave to a scanning line for the subject and receives a reflected wave.

11. The ultrasonic diagnostic apparatus of claim 9 wherein the processing circuitry is further configured to acquire the plurality of blood vessel width candidates when a second input operation is received from a user during execution of a PWD method scan after the first input operation is received, the second input operation being related to movement of the region, and the PWD method scan transmitting a pulse wave to a scanning line for the subject and receiving a reflected wave.

12. The ultrasonic diagnostic apparatus of claim 9, wherein the processing circuitry is further configured to acquire the plurality of blood vessel width candidates when a third input operation is received from the user after the first input operation is received, the third input operation being related to start of execution of a PWD method scan that transmits a pulse wave to a scanning line for the subject and receives a reflected wave.

13. The ultrasonic diagnostic apparatus of claim 1, wherein the processing circuitry is further configured to adjust the region included in the sample gate according to a ratio of the region to a blood vessel width set in advance by a user.
